(19) United States
(12) Patent Application Publication
(10) Pub. No.: US 2004/0219150 A1
(43) Pub. Date: Nov. 4, 2004

(54) Uses of mammalian cytokine; related reagents

(76) Inventors: Daniel J. Cua, Boulder Creek, CA (US); Yi Chen, Mountain View, CA (US); Robert A. Kastelein, Redwood City, CA (US); Claire L. Langrish, Palo Alto, CA (US); Donna M. Rennick, Los Altos, CA (US); Jonathon Sedgwick, Palo Alto, CA (US)

Correspondence Address: DNAX RESEARCH, INC., LEGAL DEPARTMENT, 901 CALIFORNIA AVENUE, PALO ALTO, CA, 94304, US

(21) Appl. No.: 10/773,083

(22) Filed: Feb. 4, 2004

Related U.S. Application Data
(60) Provisional application No. 60/445,592, filed on Feb. 6, 2003.
(60) Provisional application No. 60/531,342, filed on Dec. 19, 2003.

(51) Int. Cl. A61K 39/395; A61K 48/00

(52) U.S. Cl. 424/145.1; 514/44

(57) ABSTRACT

Provided are methods of treatment for inflammatory and autoimmune disorders of the central nervous system and gastrointestinal tract. Also provided are methods of diagnosis.

This application claims benefit of U.S. Provisional patent applications Ser. No. 60/445,592 filed Feb. 6, 2003, and 60/531,342, filed Dec. 19, 2003.

### FIELD OF THE INVENTION

The present invention relates generally to uses of mammalian cytokine molecules and related reagents. More specifically, the invention relates to a cytokine that mediates activities of the central nervous system.

### BACKGROUND OF THE INVENTION

Multiple sclerosis and inflammatory bowel disorders are autoimmune conditions, as autoimmune responses play a major role in these conditions. The immune system functions to protect individuals from infective agents, e.g., bacteria, multi-cellular organisms, and viruses, as well as from cancers. This system includes several types of lymphoid and myeloid cells such as monocytes, macrophages, dendritic cells (DCs), eosinophils, T cells, B cells, and neutrophils. These lymphoid and myeloid cells often produce signaling proteins known as cytokines. The immune response includes inflammation, i.e., the accumulation of immune cells systemically or in a particular location of the body. In response to an infective agent or foreign substance, immune cells secrete cytokines which, in turn, modulate immune cell proliferation, development, differentiation, or migration. Immune response can produce pathological consequences, e.g., when it involves excessive inflammation, as in the autoimmune disorders (see, e.g., Abbas, et al. (eds.) (2000) Cellular and Molecular Immunology, W. B. Saunders Co., Philadelphia, Pa.; Oppenheim and Feldmann (eds.) (2001) Cytokine Reference, Academic Press, San Diego, Calif.; von Andrian and Mackay (2000) New Engl. J. Med. 343:1020-1034; Davidson and Diamond (2001) New Engl. J. Med. 345:340-350).

Interleukin-23 (IL-23) is a heterodimeric cytokine comprised of two subunits, i.e., p19 and p40. The p19 subunit is structurally related to IL-6, granulocyte-colony stimulating factor (G-CSF), and the p35 subunit of IL-12. The p40 subunit is also part of the cytokine IL-12, which is composed of p35 and p40. IL-23 mediates signaling by binding to a heterodimeric receptor, comprised of IL-23R and IL-12beta1. The IL-12beta1 subunit is shared by the IL-12 receptor, which is composed of IL-12beta1 and IL-12beta2. A number of early studies demonstrated that the consequences of a genetic deficiency in p40 (p40 knockout mouse; p40KO mouse) were more severe than those found in a p35KO mouse. Some of these results were eventually explained by the discovery of IL-23, and the finding that the p40KO prevents expression of IL-12, but also of IL-23 (Oppmann, et al. (2000) Immunity 13:715-725; Wiekowski, et al. (2001) J. Immunol. 166:7563-7570; Parham, et al.(2002) J. Immunol 168:5699-708; Frucht (2002) Sci STKE 2002, E1-E3; Elkins, et al. (2002) Infection Immunity 70:1936-1948).

The present invention provides methods for the treatment of immune-mediated disorders of the nervous system. Macrophages and microglia are the main immune cells of the central nervous system (CNS). These cells as well as T cells, neutrophils, astrocytes, and microglia, a resident cell of the central nervous system, and having properties similar to those of monocytes and macrophages, all can contribute to the immune-related pathology of, e.g., multiple sclerosis, Alzheimer's disease, amyotrophic lateral sclerosis (ALS), ischemic brain injury, prion diseases, and HIV-associated dementia (Minagar, et al. (2002) J. Neurological Sci. 202:13-23; Antel and Owens (1999) J. Neuroimmunol. 100:181-189; Elliott (2001) Mol. Brain Res. 95:172-178; Kostulas, et al. (1999) Stroke 30:2174-2179; Nakamura (2002) Biol. Pharm. Bull. 25:945-953).

Disorders and conditions of the peripheral nervous system, e.g., neuropathic pain, posttraumatic neuropathies, Guillain-Barre syndrome (GBS), peripheral polyneuropathy, and nerve regeneration are mediated by immune cells and cytokines (see, e.g., Watkins and Maier (2002) Physiol. Rev. 82:981-1011; Veves and King (2001) J. Clin. Invest. 107:1215-1218; Snider, et al. (2002) Neuron 35:13-16).

A number of cytokines have a role in the pathology or repair of neurological disorders. IL-6, IL-17, interferon-gamma (IFNgamma), and granulocyte colony-stimulating factor (GM-CSF) have been associated with multiple sclerosis (Matusevicius, et al. (1999) Multiple Sclerosis 5:101-104; Lock, et al. (2002) Nature Med. 8:500-508). IL-1alpha, IL-1beta, and transforming growth factor-beta 1 (TGF-beta1) plays a role in ALS, Parkinson's disease, and Alzheimer's disease (Hoozemans, et al. (2001) Exp. Gerontol. 36:559-570; Griffin and Mrak (2002) J. Leukocyte Biol. 72:233-238; Ilzecka, et al. (2002) Cytokine 20:239-243). TNF-alpha, IL-1beta, IL-6, IL-8, interferon-gamma (IFNgamma), and IL-17 appear to modulate response to brain ischemia (see, e.g., Kostulas, et al. (1999) Stroke 30:2174-2179; Li, et al. (2001) J. Neuroimmunol. 116:5-14). Vascular endothelial cell growth factor (VEGF) is associated with ALS (Cleveland and Rothstein (2001) Nature 2:806-819).

Inflammatory bowel disorders, e.g., Crohn's disease, ulcerative colitis, celiac disease, and irritable bowel syndrome, are mediated by cells of the immune system and by cytokines. For example, Crohn's disease is associated with increased IL-12 and IFNgamma, while ulcerative colitis is associated with increased IL-5, IL-13, and transforming growth factor-beta (TGFbeta). IL-17 expression may also increase in Crohn's disease and ulcerative colitis (see, e.g., Podolsky (2002) New Engl. J. Med. 347:417-429; Bouma and Strober (2003) Nat. Rev. Immunol. 3:521-533; Bhan, et al. (1999) Immunol. Rev. 169:195-207; Hanauer (1996) New Engl. J. Med. 334:841-848; Green (2003) The Lancet 362:383-391; McManus (2003) New Engl. J. Med. 348:2573-2574; Horwitz and Fisher (2001) New Engl. J. Med. 344:1846-1850; Andoh, et al. (2002)Int. J. Mol. Med. 10:631-634; Nielsen, et al. (2003) Scand. J. Gastroenterol. 38:180-185; Fujino, et al. (2003) Gut 52:65-70).

There is an unmet need to treat inflammatory and immune system-mediated disorders, e.g., of the nervous system or of the gastrointestinal tract. The present invention fulfills this need by providing methods of using agonists and antagonists of a recently discovered cytokine.

### SUMMARY OF THE INVENTION

The present invention is based on the observation that an agonist or antagonist of IL-23 modulates inflammatory conditions and disorders of the nervous system and gastrointestinal tract.

The present invention provides a method of treating an IL-23 mediated disorder comprising administering an effective amount of an agonist of IL-23 or antagonist of IL-23. Also provided is the above method, wherein the disorder is a gastrointestinal disorder or nervous system disorder; or the above method wherein the agonist or antagonist specifically binds to a polypeptide or nucleic acid of p19 or IL-23R. In addition, the invention provides the above method wherein wherein the agonist or antagonist comprises a nucleic acid or small molecule; as well as the above method wherein the nucleic acid comprises anti-sense nucleic acid or small interfering RNA (siRNA).

In another embodiment, the present invention provides a method of treating an IL-23 mediated disorder comprising administering an effective amount of an agonist of IL-23 or antagonist of IL-23, wherein the agonist or antagonist is an antigen binding fragment of an antibody or a soluble receptor derived from IL-23R; or the above method wherein the agonist or antagonist a polyclonal antibody; a monoclonal antibody; a humanized antibody or binding fragment thereof; an Fab, Fv, or F(ab′)₂ fragment; a peptide mimetic of an antibody; detectably labeled.

In another aspect, the present invention provides the above method wherein the nervous system disorder is a central nervous system (CNS) disorder or peripheral nervous system (PNS) disorder; or the above method wherein the condition or disorder comprises multiple sclerosis; neuropathic pain; amyotrophic lateral sclerosis (ALS); ischemic brain injury; or inflammatory bowel disorder; as well as the above method wherein the inflammatory bowel disorder comprises Crohn's disease; ulcerative colitis; celiac disease; mucosal thickening; epithelial hyperplasia; inflammation of the submucosa or tunica muscularis; or infiltration by granulocytes or macrophages.

Yet another aspect of the present invention provides the above method, wherein the agonist or antagonist if IL-23 is co-administered with an agonist or antagonist of IL-12; interferon-gamma (IFNgamma); IL-6; IL-17; or IL-10; or the above method wherein the nervous system disorder is exacerbated by an antagonist of IL-12 or IFNgamma. Also provided is the above method wherein the nervous system disorder comprises an increase in microglial expression of IL-12Rbeta1, p19, or p40; comprises an increase of CNS macrophage expression of IL-23R, IL-12Rbeta1, IL-12Rbeta2, p 19, or p35; or can be generated in human or animal subject by administration of exogenous IL-17 producing cells to the subject.

Also encompassed is the above method, wherein the administration inhibits activation of a resident microglial cell; and the above method wherein the microglial cell is CD11b⁺CD45^{low}; or where activation comprises up-regulation of MHC-Class II. Moreover, the invention provides the above method wherein the antagonist inhibits expression of IL-1beta by a macrophage; expression of tumor necrosis factor (TNF) by a macrophage; or infiltration of a macrophage into the central nervous system (CNS). Another embodiment provides the above method, wherein the macrophage is: F4/80⁺; CD11b⁺; CD11c⁻; or B220⁻.

The present invention provides a purified or isolated IL-17 producing CD4⁺ T cell that upon treatment with IL-23 has a 10-fold higher expression of at least one gene of Table 10B, e.g., IL-17 or IL-75, when compared to treatment with IL-12; the above cell that is: CD62^{lo}CD44^{hi} or CD45RB^{lo}. Also provided is a method of generating the above IL-17 producing CD4⁺ T cell comprising contacting a T cell with a substantially pure preparation of an agonist of IL-23 or an antagonist of IL-23.

### DETAILED DESCRIPTION

As used herein, including the appended claims, the singular forms of words such as “a,” “an,” and “the,” include their corresponding plural references unless the context clearly dictates otherwise. All references cited herein are incorporated by reference to the same extent as if each individual publication, patent application, or patent, was specifically and individually indicated to be incorporated by reference.

I. Definitions.

“Activation,” “stimulation,” and “treatment,” as it applies to cells or to receptors, may have the same meaning, e.g., activation, stimulation, or treatment of a cell or receptor with a ligand, unless indicated otherwise by the context or explicitly. “Ligand” encompasses natural and synthetic ligands, e.g., cytokines, cytokine variants, analogues, muteins, and binding compositions derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Proliferative activity” encompasses an activity that promotes, that is necessary for, or that is specifically associated with, e.g., normal cell division, as well as cancer, tumors, dysplasia, cell transformation, metastasis, and angiogenesis.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an IL-23 agonist or IL-23 antagonist to a human or animal subject, a cell, tissue, physiological compartment, or physiological fluid. “Treatment of a cell” also encompasses situations where the IL-23 agonist or IL-23 antagonist contacts IL-23 receptor (IL-23R/IL-12Rbeta1 heterodimer), e.g., in the fluid phase or colloidal phase, but also situations where the agonist or antagonist does not contact the cell or the receptor.

“Binding composition” refers to a molecule, small molecule, macromolecule, antibody, a fragment or analogue thereof, or soluble receptor, capable of binding to a target. “Binding composition” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, which is capable of binding to a target. “Binding composition” may also refer to a molecule in combination with a stabilizer, excipient, salt, buffer, solvent, or additive, capable of binding to a target. “Binding” may be defined as an association of the binding composition with a target where the association results in reduction in the normal Brownian motion of the binding composition, in cases where the binding composition can be dissolved or suspended in solution.

A “classical TH1-type T cell” is a T cell that expresses interferon-gamma (IFNgamma) to an extent greater than expression of each of IL-4, IL-5, or IL-13, while a “classical TH2-type T cell” is a T cell that expresses IL-4, IL-5, or IL-13, each to an extent greater than expression of IFNgamma. “Extent” is typically 4-fold or more, more typically 8-fold or more, and most typically 16-fold or more than for a classical TH2-type cell.

“Conservatively modified variants” applies to both amino acid and nucleic acid sequences. With respect to particular nucleic acid sequences, conservatively modified variants refers to those nucleic acids which encode identical or essentially identical amino acid sequences or, where the nucleic acid does not encode an amino acid sequence, to essentially identical nucleic acid sequences. Because of the degeneracy of the genetic code, a large number of functionally identical nucleic acids may encode any given protein.

As to amino acid sequences, one of skill will recognize that an individual substitution to a nucleic acid, peptide, polypeptide, or protein sequence which substitutes an amino acid or a small percentage of amino acids in the encoded sequence for a conserved amino acid is a “conservatively modified variant.” Conservative substitution tables providing functionally similar amino acids are well known in the art. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157: 105-132):

(1) Hydrophobic: Norleucine, Ile, Val, Leu, Phe, Cys, or Met;

(2) Neutral hydrophilic: Cys, Ser, Thr;

(3) Acidic: Asp, Glu;

(4) Basic: Asn, Gln, His, Lys, Arg;

(5) Residues that influence chain orientation: Gly, Pro;

(6) Aromatic: Trp, Tyr, Phe;

(7) Small amino acids: Gly, Ala, Ser.

“Effective amount” encompasses an amount sufficient to ameliorate or prevent a symptom or sign of the medical condition. Effective amount also means an amount sufficient to allow or facilitate diagnosis. An effective amount for a particular patient or veterinary subject may vary depending on factors such as the condition being treated, the overall health of the patient, the method route and dose of administration and the severity of side affects (see, e.g., U.S. Pat. No. 5,888,530 issued to Netti, et al.). An effective amount can be the maximal dose or dosing protocol that avoids significant side effects or toxic effects. The effect will result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

“Exogenous” refers to substances that are produced outside an organism, cell, or human body, depending on the context. “Endogenous” refers to substances that are produced within a cell, organism, or human body, depending on the context.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist irradication by the immune system. “Cancerous condition” includes, e.g., cancer, cancer cells, tumors, angiogenesis, and precancerous conditions such as dysplasia.

“Inflammatory disorder” means a disorder or pathological condition where the pathology results, in whole or in part, from, e.g., a change in number, change in rate of migration, or change in activation, of cells of the immune system. Cells of the immune system include, e.g., T cells, B cells, monocytes or macrophages, antigen presenting cells (APCs), dendritic cells, microglia, NK cells, NKT cells, neutrophils, eosinophils, mast cells, or any other cell specifically associated with the immunology, for example, cytokine-producing endothelial or epithelial cells.

An “IL-17-producing cell”means a T cell that is not a classical TH1-type T cell or classical TH2-type T cell. “IL-17-producing cell” also means a T cell that expresses a gene or polypeptide of Table 10B (e.g., mitogen responsive P-protein; chemokine ligand 2; interleukin-17 (IL-17); transcription factor RAR related; and/or suppressor of cytokine signaling 3), where expression with treatment by an IL-23 agonist is greater than treatment with an IL-12 agonist, where “greater than” is defined as follows. Expression with an IL-23 agonist is ordinarily at least 5-fold greater, typically at least 10-fold greater, more typically at least 15-fold greater, most typically at least 20-fold greater, preferably at least 25-fold greater, and most preferably at least 30-fold greater, than with IL-12 treatment. Expression can be measured, e.g., with treatment of a population of substantially pure IL-17 producing cells.

Moreover, “IL-17-producing cell” includes a progenitor or precursor cell that is committed, in a pathway of cell development or cell differentiation, to differentiating into an IL-17-producing cell, as defined above. A progenitor or precursor cell to the IL-17 producing cell can be found in a draining lymph node (DLN). Additionally, “IL-17-producing cell” encompasses an IL-17-producing cell, as defined above, that has been, e.g., activated, e.g., by a phorbol ester, ionophore, and/or carcinogen, further differentiated, stored, frozen, dessicated, inactivated, partially degraded, e.g., by apoptosis, proteolysis, or lipid oxidation, or modified, e.g., by recombinant technology.

“Inhibitors” and “antagonists” or “activators” and “agonists” refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a composition that reduces, blocks, or inactivates a constitutive activity. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

To examine the extent of inhibition, for example, samples or assays comprising a given, e.g., protein, gene, cell, or organism, are treated with a potential activator or inhibitor and are compared to control samples without the inhibitor. Control samples, i.e., not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

“Knockout” (KO) refers to the partial or complete reduction of expression of at least a portion of a polypeptide encoded by a gene, e.g., encoding a subunit of IL-23 or IL-23 receptor, where the gene is endogenous to a single cell, selected cells, or all of the cells of a mammal. KO also encompasses embodiments where biological function is reduced, but where expression is not necessarily reduced, e.g., a polypeptide that contains an inserted inactivating peptide. Disruptions in a coding sequence or a regulatory sequence are encompassed by the knockout technique. The cell or mammal may be a “heterozygous knockout”, where one allele of the endogenous gene has been disrupted. Alternatively, the cell or mammal may be a “homozygous knockout” where both alleles of the endogenous gene have been disrupted. “Homozygous knockout” is not intended to limit the disruption of both alleles to identical techniques or to identical outcomes at the genome.

A composition that is “labeled” is detectable, either directly or indirectly, by spectroscopic, photochemical, biochemical, immunochemical, isotopic, or chemical methods. For example, useful labels include ³²P, ³³P, ³⁵S ¹⁴C, ³H, ¹²⁵I, stable isotopes, fluorescent dyes, electron-dense reagents, substrates, epitope tags, or enzymes, e.g., as used in enzyme-linked immunoassays, or fluorettes (see, e.g., Rozinov and Nolan (1998) Chem. Biol. 5:713-728).

“Ligand” refers, e.g., to a small molecule, peptide, polypeptide, and membrane associated or membrane-bound molecule, or complex thereof, that can act as an agonist or antagonist of a receptor. “Ligand” also encompasses an agent that is not an agonist or antagonist, but that can bind to the receptor. Moreover, “ligand” includes a membrane-bound ligand that has been changed, e.g., by chemical or recombinant methods, to a soluble version of the membrane-bound ligand. By convention, where a ligand is membrane-bound on a first cell, the receptor usually occurs on a second cell. The second cell may have the same or a different identity as the first cell. A ligand or receptor may be entirely intracellular, that is, it may reside in the cytosol, nucleus, or some other intracellular compartment. The ligand or receptor may change its location, e.g., from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

A “marker” relates to the phenotype of a cell, tissue, organ, animal, e.g., of an IL-17 producing cell. Markers are used to detect cells, e.g., during cell purification, quantitation, migration, activation, maturation, or development, and may be used for both in vitro and in vivo studies. An activation marker is a marker that is associated with cell activation.

“Purified cell” encompasses, e.g., one or more “IL-17 producing cells” that is substantially free of other types of cells, e.g., contamination by other types of T cells. Purity can be assessed by use of a volume that is defined by geometric coordinates or by a compartment comprising, e.g., a flask, tube, or vial. A “purified IL-17 producing cell” can be defined by, e.g., a compartment where the “IL-17 producing cells” normally constitute at least 20% of all the cells, more normally at least 30% of all the cells, most normally at least 40% of all the cells, generally at least 50% of all the cells, more generally at least 60% of all the cells, most generally at least 70% of all the cells, preferably at least 80% of all the cells, more preferably at least 90% of all the cells; and most preferably at least 95% of all the cells.

“Small molecules” are provided for the treatment of physiology and disorders of the hair follicle. “Small molecule” is defined as a molecule with a molecular weight that is less than 10 kD, typically less than 2 kD, and preferably less than 1 kD. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins are described (see, e.g., Casset, et al. (2003) Biochem. Biophys. Res. Commun. 307:198-205; Muyldermans (2001) J Biotechnol. 74:277-302; Li (2000) Nat. Biotechnol. 18:1251-1256; Apostolopoulos, et al. (2002) Curr. Med. Chem. 9:411-420; Monfardini, et al. (2002) Curr. Pharm. Des. 8:2185-2199; Domingues, et al. (1999) Nat. Struct. Biol. 6:652-656; Sato and Sone (2003) Biochem. J. 371:603-608; U.S. Pat. No. 6,326,482 issued to Stewart, et al).

“Specifically” or “selectively” binds, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two fold greater, preferably at least ten times greater, more preferably at least 20-times greater, and most preferably at least 100-times greater than the affinity with any other antibody, or binding composition derived thereof. In a preferred embodiment the antibody will have an affinity that is greater than about 10⁹ liters/mol, as determined, e.g., by Scatchard analysis (Munsen, et al. (1980) Analyt. Biochem. 107:220-239).

II. General.

The present invention provides methods of using polypeptides, nucleic acids, variants, muteins, and mimetics of IL-23, p19 subunit, p40 subunit, IL-23 receptor, IL-23R subunit, or IL-12Rbeta1 subunit. Also provided are methods for using a hyperkine, i.e., a fusion protein comprising, e.g., the p19 subunit linked to the p40 subunit of IL-23, as well as nucleic acids encoding the hyperkine (see, e.g., Oppmann, et al., supra; Fischer, et al. (1997) Nature Biotechnol. 15:142-145; Rakemann, et al. (1999) J. Biol. Chem. 274:1257-1266; and Peters, et al.(1998) J. Immunol. 161:3575-3581).

Administration of an IL-23 agonist, i.e., IL-23 or IL-23 hyperkine, can induce, e.g., proliferation of memory T cells, PHA blasts, CD45RO T cells, CD45RO T cells; enhance production of interferon-gamma (IFNgamma) by PHA blasts or CD45RO T cells. In contrast to IL-12, IL-23 preferentially stimulates memory as opposed to naive T cell populations in both human and mouse. IL-23 activates a number of intracellular cell-signaling molecules, e.g., Jak2, Tyk2, Stat1, Stat2, Stat3, and Stat4. IL-12 activates this same group of molecules, but Stat4 response to IL-23 is relatively weak, while Stat4 response to IL-12 is strong (Oppmann, et al., supra; Parham, et al. (2002) J. Immunol. 168:5699-5708).

Administration of the p 19 subunit of IL-23 can result in, e.g., stunted growth, infertility, and death of animals, as well as inflammatory infiltrates, e.g., in the gastrointestinal tract, lungs, skin, and liver, and epithelial cell hyperplasia, microcytic anemia, increased neutrophil count, increased serum tumor necrosis factor-alpha (TNFalpha); and increased expression of acute phase genes in liver (Wiekowski, et al., supra).

Other studies have demonstrated that IL-23 modulates immune response to infection (see, e.g., Pirhonen, et al. (2002) J. Immunol. 169:5673-5678; Broberg, et al. (2002) J. Interferon Cytokine Res. 22:641-651; Elkins, et al. (2002) Infection Immunity 70:1936-1948; Cooper, et al. (2002) J. Immunol. 168:1322-1327).

III. Agonists, Antagonists, and Binding Compositions.

Agonists of IL-23 encompass, e.g., IL-23, an IL-23 variant, mutein, or peptide mimetic, agonistic antibodies to IL-23 receptor, and nucleic acids encoding these agonists. Antagonists of IL-23 include, e.g., antibodies to IL-23, blocking antibodies to IL-23 receptor, a soluble receptor based on the extracellular region of a subunit of the IL-23 receptor, peptide mimetics thereto, and nucleic acids encoding these antagonists. Binding compositions that specifically bind to p19 of IL-23 or to IL-23R of IL-23 receptor are provided.

Regions of increased antigenicity can be used for antibody generation. Regions of increased antigenicity of human p19 occur, e.g., at amino acids 16-28; 57-87; 110-114; 136-154; and 182-186 of GenBank AAQ89442 (gi:37183284). Regions of increased antigenicity of human IL-23R occur, e.g., at amino acids 22-33; 57-63; 68-74; 101-112; 117-133; 164-177; 244-264; 294-302; 315-326; 347-354; 444-473; 510-530; and 554-558 of GenBank AAM44229 (gi: 21239252). Analysis was by a Parker plot using Vector NTI® Suite (Informax, Inc, Bethesda, Md.). The present invention also provides an EL-23 antagonist that is a soluble receptor, i.e., comprising an extracellular region of IL-23R, e.g., amino acids 1-353 of GenBankAAM44229, or a fragment thereof, where the extracellular region or fragment thereof specifically binds to IL-23. Mouse IL-23R is GenBankNP_{—}653131 (gi:21362353). Muteins and variants are contemplated, e.g., pegylation or mutagenesis to remove or replace deamidating Asn residues.

An agonist or antagonist of an IL-17 producing cell encompasses a reagent that specifically modulates the activity of an IL-17 producing cell, e.g., without substantial influence on the activity of, e.g., a naive T cell, TH1-type T cell, TH2-type T cell, epithelial cell, and/or endothelial cell. The reagent can modulate expression or activity of, e.g., a transcription factor or adhesion protein, of the IL-17 producing cell.

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, N.Y.; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang, et al. (1999) J. Biol. Chem. 274:27371-27378; Baca, et al. (1997) J. Biol. Chem. 272:10678-10684; Chothia, et al. (1989) Nature 342:877-883; Foote and Winter (1992) J. Mol. Biol. 224:487-499; U.S. Pat. No. 6,329,511 issued to Vasquez, et al.).

Purification of antigen is not necessary for the generation of antibodies. Immunization can be performed by DNA vector immunization, see, e.g., Wang, et al. (1997) Virology 228:278-284. Alternatively, animals can be immunized with cells bearing the antigen of interest. Splenocytes can then be isolated from the immunized animals, and the splenocytes can fused with a myeloma cell line to produce a hybridoma (Meyaard, et al. (1997) Immunity 7:283-290; Wright, et al. (2000) Immunity 13:233-242; Preston, et al. (1997) Eur. J. Immunol. 27:1911-1918). Resultant hybridomas can be screened for production of the desired antibody by functional assays or biological assays, that is, assays not dependent on possession of the purified antigen. Immunization with cells may prove superior for antibody generation than immunization with purified antigen (Kaithamana, et al. (1999) J. Immunol. 163:5157-5164).

Antibody to antigen and ligand to receptor binding properties can be measured, e.g., by surface plasmon resonance (Karlsson, et al. (1991) J. Immunol. Methods 145:229-240; Neri, et al. (1997) Nat. Biotechnol. 15:1271-1275; Jonsson, et al. (1991) Biotechniques 11:620-627) or by competition ELISA (Friguet, et al. (1985) J. Immunol. Methods 77:305-319; Hubble (1997) Immunol. Today 18:305-306). Antibodies can be used for affinity purification to isolate the antibody's target antigen and associated bound proteins, see, e.g., Wilchek, et al. (1984) Meth. Enzymol. 104:3-55.

Antibodies will usually bind with at least a K_{D} of about 10⁻³ M, more usually at least 10⁻⁶ M, typically at least 10⁻⁷ M, more typically at least 10⁻⁸ M, preferably at least about 10⁻⁹ M, and more preferably at least 10⁻¹⁰ M, and most preferably at least 10⁻¹¹ M (see, e.g., Presta, et al. (2001) Thromb. Haemost. 85:379-389; Yang, et al. (2001) Crit. Rev. Oncol. Hematol. 38:17-23; Carnahan, et al. (2003) Clin. Cancer Res. (Suppl.) 9:3982s-3990s).

Soluble receptors comprising the extracellular domains of IL-23R or IL-12Rbeta1 receptor polypeptides are provided. Soluble receptors can be prepared and used according to standard methods (see, e.g., Jones, et al. (2002) Biochim. Biophys. Acta 1592:251-263; Prudhomme, et al. (2001) Expert Opinion Biol. Ther. 1:359-373; Fernandez-Botran (1999) Crit. Rev. Clin. Lab Sci. 36:165-224).

IV. Therapeutic Compositions, Methods.

The invention provides IL-23 and anti-IL-23R for use, e.g., in the treatment of inflammatory and autoimmune disorders. Nucleic acids are also provided for these therapeutic uses, e.g., nucleic acids encoding IL-23 or IL-23R, or an antigenic fragment thereof, the corresponding anti-sense nucleic acids, and hybridization products thereof. The invention also provides compositions for RNA interference (see, e.g., Arenz and Schepers (2003) Naturwissenschaften 90:345-359; Sazani and Kole (2003) J. Clin. Invest. 112:481-486; Pirollo, et al. (2003) Pharmacol. Therapeutics 99:55-77; Wang, et al. (2003) Antisense Nucl. Acid Drug Devel. 13:169-189.

To prepare pharmaceutical or sterile compositions including an agonist or antagonist of IL-23, the cytokine analogue or mutein, antibody thereto, or nucleic acid thereof, is admixed with a pharmaceutically acceptable carrier or excipient, see, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with physiologically acceptable carriers, excipients, or stabilizers in the form of, e.g., lyophilized powders, slurries, aqueous solutions or suspensions (see, e.g., Hardman, et al. (2001) Goodman and Gilman 's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms. Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

The route of administration is by, e.g., topical or cutaneous application, injection or infusion by intravenous, intraperitoneal, intracerebral, intramuscular, intraocular, intraarterial, intracerebrospinal, intralesional, or pulmonary routes, or by sustained release systems or an implant. Injection of gene transfer vectors into the central nervous system has been described (see, e.g., Cua, et al. (2001) J. Immunol. 166:602-608; Sidman et al. (1983) Biopolymers 22:547-556; Langer, et al. (1981) J. Biomed. Mater. Res. 15:167-277; Langer (1982) Chem. Tech. 12:98-105; Epstein, et al. (1985) Proc. NatL. Acad. Sci. USA 82:3688-3692; Hwang, et al. (1980) Proc. Natl. Acad. Sci. USA 77:4030-4034; U.S. Pat. Nos. 6,350466 and 6,316,024).

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Antibodies, antibody fragments, and cytokines can be provided by continuous infusion, or by doses at intervals of, e.g., one day, one week, or 1-7 times per week. Doses may be provided intravenously, subcutaneously, topically, orally, nasally, rectally, intramuscular, intracerebrally, intraspinally, or by inhalation. A preferred dose protocol is one involving the maximal dose or dose frequency that avoids significant undesirable side effects. A total weekly dose is generally at least 0.05 μg/kg body weight, more generally at least 0.2 μg/kg, most generally at least 0.5 μg/kg, typically at least 1 μg/kg, more typically at least 10 μg/kg, most typically at least 100 μg/kg, preferably at least 0.2 mg/kg, more preferably at least 1.0 mg/kg, most preferably at least 2.0 mg/kg, optimally at least 10 mg/kg, more optimally at least 25 mg/kg, and most optimally at least 50 mg/kg (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). The desired dose of a small molecule therapeutic, e.g., a peptide mimetic, natural product, or organic chemical, is about the same as for an antibody or polypeptide, on a moles/kg basis.

An effective amount for a particular patient may vary depending on factors such as the condition being treated, the overall health of the patient, the method route and dose of administration and the severity of side affects, see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK.

Typical veterinary, experimental, or research subjects include monkeys, dogs, cats, rats, mice, rabbits, guinea pigs, horses, and humans.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

Methods for co-administration or treatment with a second therapeutic agent, e.g., a cytokine, steroid, chemotherapeutic agent, antibiotic, or radiation, are well known in the art, see, e.g., Hardman, et al. (eds.) (2001) Goodman and Gilman 's The Pharmacological Basis of Therapeutics, 10^{th} ed., McGraw-Hill, New York, N.Y.; Poole and Peterson (eds.) (2001) Pharmacotherapeutics for Advanced Practice: A Practical Approach, Lippincott, Williams & Wilkins, Phila., PA; Chabner and Longo (eds.) (2001) Cancer Chemotherapy and Biotherapy, Lippincott, Williams & Wilkins, Phila., PA. An effective amount of therapeutic will decrease the symptoms typically by at least 10%; usually by at least 20%; preferably at least about 30%; more preferably at least 40%, and most preferably by at least 50%.

V. Kits and Diagnostic Reagents.

This invention provides IL-23 proteins, fragments thereof, nucleic acids, and fragments thereof, in a diagnostic kit. Also provided are binding compositions, including antibodies or antibody fragments, for the detection of IL-23 and IL-23 receptor, and metabolites and breakdown products thereof. Typically, the kit will have a compartment containing either a p 19 polypeptide, or an antigenic fragment thereof, a binding composition thereto, or a nucleic acid, e.g., a nucleic acid probe or primer.

The kit may comprise, e.g., a reagent and a compartment, a reagent and instructions for use, or a reagent with a compartment and instructions for use. The reagent may comprise an IL-23 or IL-23R, or an antigenic fragment thereof, a binding composition, or a nucleic acid. A kit for determining the binding of a test compound, e.g., acquired from a biological sample or from a chemical library, can comprise a control compound, a labeled compound, and a method for separating free labeled compound from bound labeled compound.

Diagnostic assays can be used with biological matrices such as live cells, cell extracts, cell lysates, fixed cells, cell cultures, bodily fluids, or forensic samples. Conjugated antibodies useful for diagnostic or kit purposes, include antibodies coupled to dyes, isotopes, enzymes, and metals (see, e.g., Le Doussal, et al. (1991) New Engl. J. Med. 146:169-175; Gibellini, et al. (1998) J. Immunol. 160:3891-3898; Hsing and Bishop (1999) New Engl. J Med. 162:2804-2811; Everts, et al. (2002) New Engl. J. Med. 168:883-889). Various assay formats exist, such as radioimmunoassays (RIA), ELISA, and lab on a chip (U.S. Pat. Nos. 6,176,962 and 6,517,234).

This invention provides polypeptides and nucleic acids of IL-23 and IL-23R, fragments thereof, in a diagnostic kit, e.g., for the diagnosis of inflammatory disorders of the central and peripheral nervous system, and gastrointestinal tract.

Also provided are binding compositions, including antibodies or antibody fragments, for the detection of IL-23 and IL-23R and metabolites and breakdown products thereof. Typically, the kit will have a compartment containing either a IL-23 or IL-23R polypeptide, or an antigenic fragment thereof, a binding composition thereto, or a nucleic acid, such as a nucleic acid probe, primer, or molecular beacon (see, e.g., Rajendran, et al. (2003) Nucleic Acids Res. 31:5700-5713; Cockerill (2003) Arch. Pathol. Lab. Med. 127:1112-1120; Zammatteo, et al. (2002) Biotech. Annu. Rev. 8:85-101; Klein (2002) Trends Mol. Med. 8:257-260).

A method of diagnosis can comprise contacting a sample from a subject, e.g., a test subject, with a binding composition that specifically binds to a polypeptide or nucleic acid of IL-23 or IL-23R. The method can further comprise contacting a sample from a control subject, normal subject, or normal tissue or fluid from the test subject, with the binding composition. Moreover, the method can additionally comprise comparing the specific binding of the composition to the test subject with the specific binding of the composition to the normal subject, control subject, or normal tissue or fluid from the test subject. Expression or activity of a test sample or test subject can be compared with that from a control sample or control subject. A control sample can comprise, e.g., a sample of non-affected or non-inflamed tissue in a patient suffering from an immune disorder. Expression or activity from a control subject or control sample can be provided as a predetermined value, e.g., acquired from a statistically appropriate group of control subjects.

The kit may comprise, e.g., a reagent and a compartment, a reagent and instructions for use, or a reagent with a compartment and instructions for use. The reagent may comprise an agonist or antagonist of IL-23 or IL-23R, or an antigenic fragment thereof, a binding composition, or a nucleic acid in a sense and/or anti-sense orientation. A kit for determining the binding of a test compound, e.g., acquired from a biological sample or from a chemical library, can comprise a control compound, a labeled compound, and a method for separating free labeled compound from bound labeled compound.

Diagnostic assays can be used with biological matrices such as live cells, cell extracts, cell lysates, fixed cells, cell cultures, bodily fluids, or forensic samples. Conjugated antibodies useful for diagnostic or kit purposes, include antibodies coupled to dyes, isotopes, enzymes, and metals (see, e.g., Le Doussal, et al. (1991) New Engl. J. Med. 146:169-175; Gibellini, et al. (1998) J. Immunol. 160:3891-3898; Hsing and Bishop (1999) New Engl. J. Med. 162:2804-2811; Everts, et al. (2002) New Engl. J Med. 168:883-889). Various assay formats exist, such as radioimmunoassays (RIA), ELISA, and lab on a chip (U.S. Pat. Nos. 6,176,962 and 6,517,234).

VI. Uses.

The present invention provides methods for using agonists and antagonists of IL-23 for the treatment and diagnosis of inflammatory disorders and conditions, e.g., of the central nervous system, peripheral nervous system, and gastrointestinal tract.

Methods are provided for the treatment of, e.g., multiple sclerosis (MS), including relapsing-remitting MS and primary progressive MS, Alzheimer's disease, amyotrophic lateral sclerosis (a.k.a. ALS; Lou Gehrig's disease), ischemic brain injury, prion diseases, and HIV-associated dementia. Also provided are methods for treating neuropathic pain, posttraumatic neuropathies, Guillain-Barre syndrome (GBS), peripheral polyneuropathy, and nerve regeneration.

Provides are methods for treating or ameliorating one or more of the following features, symptoms, aspects, manifestations, or signs of multiple sclerosis, or other inflammatory disorder or condition of the nervous system: brain lesions, myelin lesions, demyelination, demyelinated plaques, visual disturbance, loss of balance or coordination, spasticity, sensory disturbances, incontinence, pain, weakness, fatigue, paralysis, cognitive impairment, bradyphrenia, diplopia, optic neuritis, paresthesia, gait ataxia, fatigue, Uhtoff's symptom, neuralgia, aphasia, apraxia, seizures, visual-field loss, dementia, extrapyramidal phenomena, depression, sense of well-being, or other emotional symptoms, chronic progressive myelopathy, and a symptom detected by magnetic resonance imaging (MRI), including gadolinium-enhancing lesions, evoked potential recordings, or examination of cerebrospinal fluid (see, e.g., Kenealy, et al. (2003) J. Neuroimmunol. 143:7-12; Noseworthy, et al. (2000) New Engl. J. Med. 343:938-952; Miller, et al. (2003) New Engl. J. Med. 348:15-23; Chang, et al. (2002) New Engl. J. Med. 346:165-173; Bruck and Stadelmann (2003) Neurol. Sci. 24 Suppl.5:S265-S267).

The present invention also provides methods for the treatment and diagnosis of neuropathic pain, a disorder that can involve demyelination. Neuropathic pain can present with negative symptoms or positive symptoms. Negative symptoms include diminished sensitivity to pain or stimulation (hypoalgesia and hypoesthesia), while positive symptoms include spontaneous sensations (stimulus independent), e.g., burning or numbness. Positive symptoms also include evoked sensations, that is, increased response to stimuli that is ordinarily painful, such as heating and mechanical stimuli (hyperalgesia), and increased response to stimuli that is ordinarily not painful, such warming, mild, cooling, or touch (allodynia). Neuropathic pain can result from a primary insult to the peripheral or central nervous system. The primary insult can take the form of immune inflammation, e.g., multiple sclerosis, mechanical injury, diabetes, a virus, chemotherapy, or ischemia. Cytokines, such as IL-1 beta, TNFalpha, IL-6, CXCL8, and CXCL5, appear to be involved neuropathic pain (see, e.g., Vrinten, et al. (2001) Euro. J. Pharmacol. 429:61-69; Zimmerman (2001) Euro. J. Pharmacol. 429:23-37; Boddeke (2001) Euro. J. Pharmacol. 429:115-119; Rutkowski and DeLeo (2002) Drug News Perspect. 15:626-632; Lindenlaub and Sommer (2003) Acta Neuropathol. (Berl.) 105:593-602; Sommer (2003) Curr. Opin. Neurol. 16:623-628; Calcutt (2002) Int. Rev. Neurobiol. 50:205-228; Levy (1996) New Engl. J. Med. 335:1124-1132).

Moreover, the present invention provides methods for treating and diagnosing inflammatory bowel disorders, e.g., Crohn's disease, ulcerative colitis, celiac disease, and irritable bowel syndrome. Provides are methods for treating or ameliorating one or more of the following symptoms, aspects, manifestations, or signs of an inflammatory bowel disorder: malabsorption of food, altered bowel motility, infection, fever, abdominal pain, diarrhea, rectal bleeding, weight loss, signs of malnutrition, perianal disease, abdominal mass, and growth failure, as well as intestinal complications such as stricture, fistulas, toxic megacolon, perforation, and cancer, and including endoscopic findings, such as, friability, aphthous and linear ulcers, cobblestone appearance, pseudopolyps, and rectal involvement and, in addition, anti-yeast antibodies (see, e.g., Podolsky, supra; Hanauer, supra; Horwitz and Fisher, supra).

The broad scope of this invention is best understood with reference to the following examples, which are not intended to limit the inventions to the specific embodiments.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

### EXAMPLES

I. General Methods.

Standard methods in molecular biology are described (Maniatis, et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Productsfor Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies is described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protcols in Immunology, Vol. 4, John Wiley, Inc., New York).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

II. Isolation of Cells and Gene Expression.

Methods of the present invention for the study of EAE were as follows. T cells, macrophages, and resident microglia were isolated by digesting brain/spinal cord homogenate with collagenase and DNAse followed by Percoll® gradient centrifugation (Sedgwick, et al., supra). The number of CD4⁺CD45^{hi} T cells, CD4⁻ CD11b⁺ CD45^{hi} inflammatory macrophages, and CD4⁻CD11b⁺CD45^{low} resident microglia in the CNS was determined by multiplying the percent of lineage-marker positive cells by the total number of mononuclear cells isolated from the CNS. Inflammatory macrophages and resident microglia were isolated as distinct populations from the CNS of CD45.1 B6 congenic donor bone-marrow cells. The CD11b⁺CD45.1^{hi}CD45.2⁻ inflammatory macrophages and CD11b⁺CD45.2^{low}CD45.1⁻ resident microglia (radiation resistant cells remaining of the host CD45 allotype) were purified by 3 color flow cytometry from >100 irradiation bone-marrow chimeric mice. At various time points after EAE induction, purified and sorted cells were pooled (microglia or inflammatory macrophages) and analyzed by quantitative real-time PCR for IL-12 and IL-23 ligand and receptor subunits.

RNA from tissues or cell pellets was extracted using RNeasy® columns (Qiagen, Valencia, Calif.) and treated with Dnase I (Promega, Madison, Wis.). cDNA were prepared as described and used as templates for quantitative PCR. cDNA (25 ng) was analyzed for expression of a range of genes using GeneAmp® 5700 Sequence Detection System (Applied Biosystems, Foster City, Calif.). Analysis of cDNA samples from spinal cords, draining lymph nodes, or peritoneal macrophages was normalized to expression of the housekeeping gene, ubiquitin.

III. Stimulation of Macrophages, In Vivo.

Mice received injection (i.p.) of IL-12 or IL-23 in 100 microliters of PBS. Where indicated, mice were pretreated with 1 mg of neutralizing rat anti-mouse IFNgamma antibody (XMG1.2) or rat anti-beta-gal antibody (isotype control, rat IgG1) 1 hour before cytokine treatment. For IL-23 blocking studies, mice were injected (i.p.) with 10 molar excess of neutralizing rat anti-mouse p40 (C17.8) or rat anti-beta-gal mAb (isotype control, rat IgG2a) at the time of IL-23 administration. Three hours after cytokine treatment, peritoneal macrophages F4/80⁺ (CalTag, Burlingame, Calif.), CD11b⁺ (mAb M1/70), CD11c⁻ (mAb HL3), and CD45R/B220⁻ (mAb RA3-6B2, BD Biosciences, San Jose, Calif.) were isolated by multi-color flow cytometry and prepared for real time quantitative PCR analysis.

IV. Induction of EAE in Mice.

p35KO mice (a.k.a. IL-12p35 deficient mice) and p40KO mice were from The Jackson Laboratory (Bar Harbor, Me.) and had originally been generated on the B6×129 background and were back-crossed 11 generations onto the C57BL/6 background. The p19KO mice (a.k.a. p19IL-23 deficient mice) were generated and p19KO, p19^{+/-} and wild-type controls were maintained on a mixed B6×129 F2 background. EAE was induced with MOG33-55 peptide in complete Freund's adjuvant (CFA) plus pertussis toxin (Chen, et al. (2001) J Immunol 166, 3362-3368). Immunohistochemistry of fresh spinal cord tissues was performed. Draining lymph node (DLN) cells were isolated 6 days after immunization and T cell proliferation and cytokine secretion assays, e.g., ELISA, were performed. In studies with p19KO, p35KO, and p40KO mice, p40KO mice, which lack both IL-12 and IL-23, and p19KO mice, which lack IL-23, were EAE resistant. p35KO mice that specifically lacked IL-12, by contrast, were highly susceptible to EAE. Intense mononuclear cell infiltration of the spinal cord was observed in control heterozygous p19^{+/-} and p35KO mice but not in p19KO or p40KO mice. Thus, IL-23 but not IL-12 is critical for the development of CNS autoimmune inflammation.

V. Mice Deficient in IL-23 Resist EAE, Intracerebral IL-23 Reconstitutes EAE.

IL-23 and IL-23-rAdV are described (Oppmann, et al., supra). For CNS administration, recombinant cytokines or rAdV gene transfer vectors were suspended in 10 microliters of phosphate buffered saline (PBS) and injected into the right-lateral cerebral ventricle using a 28-gauge needle as described (Cua, et al., supra). For peripheral delivery, cytokines or rAdV vectors suspended in 100 microliters of PBS were injected into the tail vein. The vector was injected 2 days before expected onset of EAE.

IL-23 expressed in the CNS, by way of the vector, reconstituted or reinstated EAE in both p 19KO and p40KO mice, although the p40KO mice had delayed disease onset and reduced disease severity. Systemic expression of IL-23 alone was not sufficient to enable disease induction. A control intracerebral injection of control adenovirus expressing only a green-fluorescence protein (GFP) gene had no effect.

VI. Relative Contributions of IL-23 and IL-12 to EAE.

The relative contributions of administered IL-23 and IL-12 to EAE development in p40KO mice was studied. IL-12 or IL-12 plus IL-23 was administered to p40KO mice during disease induction.

Treatment of p40KO mice with recombinant IL-12 (i.p.) from day 0 to day 18 did not induce or reconstitute EAE. Also, IL-12-rAdV (i.c.) at day 8 did not induce or reconstitute EAE. When p40KO mice were administered IL-12 (i.p.) from day 0 to day 7 followed by IL-23 gene transfer (i.c.) at day 8, intense EAE, comparable to that found in wild type controls, was induced. Thus, IL-12 promotes development of Th1 cells, whereas IL-23 is necessary for subsequent CNS inflammatory events. These subsequent CNS events could include recruitment and/or reactivation of T cells within the CNS, or activation of inflammatory and CNS resident macrophages.

The development of MOG-specific T cells in IL-23 p19KO mice, the recruitment of T cells to the CNS of these mice as well as the effects of IL-23 on CNS resident macrophages (microglia) and perivascular/inflammatory macrophages was analyzed. Six days after MOG immunization, draining lymph node (DLN) cells from WT, p19+/−, p19KO, p35KO, and p40KO mice had equivalent antigen-specific proliferative responses regardless of their EAE susceptibility. DLN cells from p19KO mice, in vitro secreted wild type levels of IFNgamma but little or no IL-4 in response to MOG stimulation, indicating that classic Th1 cells were induced in these mice. In contrast, the response in MOG-immunized p35KO and p40KO mice was restricted to a Th2 phenotype with low IFNgamma and high IL-4 levels, consistent with the important role of IL-12 in IFNgamma production and Th1 development (O'Garra and Arai (2000) Trends Cell Biol 10, 542-550; Caspi (1998) Clin Immunol Immunopathol 88, 4-13; Falcone and Sarvetnick (1999) Curr Opin Immunol 11, 670-676). That p35KO mice with low IFNgamma are EAE susceptible, possibly even more severely affected than WT mice, is consistent with data showing that IFNgamma deficient mice have a hyper-acute form of EAE (Willenborg (1996) J. Immunol. 157:3223-3227; Chu, et al. (2000) J. Exp. Med. 192:123-128; Matthys, et al. (2001) Trends Immunol. 22:367-371).

The severity of EAE was determined by a disease score or grade. The grade of EAE was determined in wild type mice, partial knockout mice (p19^{+/-}), or in p19KO mice (p19^{-/-}) (Table 1). As p19 is a subunit that occurs uniquely in the IL-23 heterodimer, and not, e.g., in the IL-12 heterodimer, a p19KO mouse may also be called an “IL-23KO mouse.” IL-23 knockout mice (IL-23KO mice) were completely resistant to hind limb paralysis and weight loss associated with CNS inflammation, whereas both the wild type B/6×129 control mice and the p19^{+/-} (p19 subunit of IL-23) mice were highly susceptible to EAE.

Immunohistochemical staining of spinal cords from the p19^{+/-} mice showed extensive infiltration of CD11b positive cells into the CNS parenchyma, whereas no infiltrating cells were found in the p19KO mice (a.k.a. p19^{-/-} mice).

The grade of EAE found in p19KO mice, p19+/−mice, and wild type B/6×129 mice are shown (Table 1). CD11b IHC staining shows macrophage invasion of the CNS of the p 19^{+/-} mouse, but little or no macrophage invasion of the p 19KO mouse.

TABLE 1
Grade of EAE versus time after MOG immunization
in three strains of mice.

| Day | 8 | 9 | 10 | 11 | 12 | 13 | 14 | 15 | 16 |
|---|---|---|---|---|---|---|---|---|---|
| p19KO | 0 | 0 | 0 | 0 | 0 | 0 | 0 | 0 | 0 |
| p19^{+/-} | 0 | 0 | 0.5 | 1.5 | 3.7 | 4.7 | 4.5 | 4.8 | 5.0 |
| wild type | 0 | 0 | 0.4 | 2.0 | 3.5 | 4.8 | 5.2 | 4.8 | 4.4 |

Methodology in producing and evaluating EAE was as follows. Mice were immunized with 50 micrograms of MOG 35-55 peptide in complete Freund's adjuvant (CFA). At day 0 and 2, mice were injected (i.v.) with 100 ng of pertussis toxin. The p 19KO mice were on the B6×129 genetic background. Clinical status was scored as normal appearance (grade 0); limp tail (grade 1); hind limb weakness (grade 2); hind limb paralysis (grade 3); paraplegia, incontinence (grade 4); wasting, quadriplegia (grade 5); moribund (grade 6) (see Table 1).

EAE was compared in mice specifically missing IL-12 (p35KO mice), mice specifically missing IL-23 (p19KO mice), or in mice missing both IL-12 and IL-23 (p40KO mice). IL-23 consists of p19 plus p40. IL-12 consists of p35 plus p40. The p40 subunit is a common subunit of IL-12 and in IL-23. The p40KO and p19KO each provided complete protection under the conditions of study, demonstrating that IL-23 but not IL-12 plays a key role for the development of EAE (Table 2).

TABLE 2
Grade of EAE versus time after MOG immunization in
IL-23 deficient mice versus IL-12 deficient mice. N.D. means
not determined. Mice were immunized with MOG-CFA and
injected (i.v.) with pertussis toxin at days 0 and 2.

| Mouse/Day | 8 | 9 | 10 | 11 | 12 | 13 | 14 | 15 | 16 | 17 | 18 |
|---|---|---|---|---|---|---|---|---|---|---|---|
| p19KO | 0 | 0 | 0 | 0 | 0 | 0 | 0 | 0 | 0 | 0 | 0 |
| p19^{+/-} | 0 | 0.5 | 1.5 | 3.7 | 4.8 | 4.5 | 4.9 | 5.0 | N.D. | N.D. | N.D. |
| wild type | 0 | 0 | 0.4 | 2.0 | 4.0 | 4.5 | 5.0 | 5.5 | N.D. | N.D. | N.D. |
| B/6 | | | | | | | | | | | |
| p35KO | 0 | 0 | 0 | 3.0 | 5.3 | 6.2 | 6.6 | 7.1 | N.D. | N.D. | N.D. |
| p40KO | 0 | 0 | 0 | 0 | 0 | 0 | 0 | 0 | 0 | 0 | 0 |

The p19 subunit of IL-23 was expressed in wild type mice and in p35KO mice and the location of p19 expression was determined. Immunohistochemical staining was performed on spinal cords 4 days after onset of EAE. Sections of spinal cords from representative wild type controls or p35KO mice were stained with anti-CD11b (Mac1) or anti-p19 antibody. The specificity of anti-p19 antibody was confirmed by absence of immunostaining when the anti-p19 mAb was pre-absorbed with 10-fold excess of rIL-23. The p19 subunit was expressed in spinal cords of wild type mice and p35KO mice, where p19 expression was co-localized with infiltrating CD11b positive macrophages.

VII. Classical IFNgamma Producing TH1-Type T Cells do not Contribute to EAE.

The p19KO allows or permits production of IFNgamma, but prevents the EAE. The p35KO prevents production of IFNgamma, but allows or permits development of EAE. Therefore, the EAE can be prevented even in the presence of IFNgamma, and EAE is not prevented by impairing IFNgamma production. Thus, the classical IFNgamma producing TH1-type T cells do not contribute to the pathology of EAE (Table 3).

p19KO mice produced a strong in vivo IFNgamma, IL-1beta, TNF, IL-6, and GM-CSF response comparable to WT controls, whereas p40KO mice showed no induction of these proinflammatory cytokines. According to PCR analysis of DLNs from MOG-immunized mice, DLNs from p35KO mice expressed elevated levels of IL-1beta, TNF, IL-6, GM-CSF, as well as p19 subunit of IL-23, but not IFNgamma. Thus, the absence of both IL-12 and IL-23 did not prevent T cell proliferative response but otherwise resulted in profound immune unresponsiveness at multiple levels, including Th1 development and resistance to EAE induction. Absence of IL-23 alone did not prevent development or expression of Th1-associated proinflammatory cytokines, but did prevent EAE. These results indicate that IL-23 is required for steps of disease development subsequent to initial T cell activation.

Secretion of cytokine polypeptide from DLN cells from mice after 6 days of MOG immunization, and culture of the cells for 60 hours with MOG peptide, with testing of secreted cytokine polypeptide is shown (Table 4). Also tested was expression of cytokine mRNA in DLN cells from mice immunized with MOG (6 days), and cytokine mRNA in DLN cells from naïve mice (Table 5).

TABLE 3
Cell proliferation (³H incorp.) versus concentration of
MOG peptide (μg/ml).

| | Concentration of MOG peptide | | | | |
|---|---|---|---|---|---|
| Strain of | 0 μg/ml | 0.3 μg/ml | 10 μg/ml | 30 μg/ml | 100 μg/ml |
| mouse | Cell proliferation (³H c.p.m. × 10⁻³) | | | | |
| B6 wild | 8-10 × 10³ | 30 | 33 | 39 | 38 |
| type | | | | | |
| p19^{+/-} | 8-10 × 10³ | 28 | 48 | 47 | 47 |
| p19KO | 8-10 × 10³ | 33 | 48 | 50 | 55 |
| p35KO | 8-10 × 10³ | 28 | 48 | 46 | 46 |
| p40KO | 8-10 × 10³ | 28 | 40 | 50 | 55 |

TABLE 4
Secretion of cytokine polypeptide from DLN cells with culture
of the cells for 60 hours with MOG peptide.

| Source of cells | IFNgamma (ng/ml) | IL-4 (ng/ml) |
|---|---|---|
| B6 wild type | 49 | 4.8 |
| p19^{+/-} | 43 | not detected |
| p19KO | 52 | not detected |
| p35KO | 12 | 10 |
| p40KO | 3 | 14.4 |

TABLE 5
Expression of various cytokines by
Taqman ® real time PCR relative to ubiquitin (1.0).

| | | IL- | | | p19 | | GM- |
|---|---|---|---|---|---|---|---|
| Source of cells | | 1beta | IFNgamma | TNF | (of IL-23) | IL-6 | CSF |
| B6 wild | naive | 400 | 180 | 1500 | 35 | 50 | 60 |
| type | +MOG | 1200 | 450 | 3400 | 110 | 210 | 160 |
| p19^{+/-} | naive | 200 | 160 | 1800 | 20 | 30 | 40 |
| | +MOG | 4900 | 520 | 5900 | 150 | 600 | 240 |
| p19KO | naive | 1100 | 125 | 2250 | 5 | 60 | 65 |
| | +MOG | 5400 | 590 | 3500 | 3 | 950 | 160 |
| p35KO | naive | 250 | 140 | 1900 | 35 | 30 | 35 |
| | +MOG | 3100 | 75 | 6000 | 190 | 300 | 275 |
| p40KO | naive | 500 | 160 | 1800 | 35 | 30 | 65 |
| | +MOG | 800 | 80 | 2000 | 20 | 100 | 30 |

VIII. Absence of IL-23 Prevents Activation of Immune Cells within the CNS.

In the absence of IL-23, Th1 cells were able to enter the CNS, but their presence did not lead to further recruitment of T cells, macrophages, or activation of resident microglia. First, it was determined if activated T cells from p19KO mice could infiltrate the CNS. During EAE pathogenesis, CD4⁺ T cells and CD11b⁺ monocytes enter the CNS well before the onset of clinical disease (Hickey, et al. (1991) J. Neurosci. Res. 28, 254-60; Sedgwick, et al. (1991) Proc. Natl. Acad. Sci. USA 88:7438-7442). These infiltrating cells are characterized by high CD45 expression whereas resident brain macrophages (or microglia) are CD11b⁺CD45 low (Sedgwick, et al., supra).

Six days after MOG immunization, elevated and comparable numbers of CD4⁺ T cells and CD11b⁺ macrophages were recovered from the whole CNS of both p19KO and wild type control mice. Real time PCR analysis of spinal cord mRNA showed comparable expression of LT-alpha, GM-CSF, CD40L, LFA-1, P-selectin, and CCR2 mRNA 9 days after immunization (transcripts that are expressed predominantly by T cells during invasion into the CNS) in both the p 19KO and wild type mice.

Twelve days after immunization, inflamed spinal cords from wild type mice with EAE exhibited a 200- to 250-fold increase in CD4⁺ cells and CD4⁻ CD11b⁺ CD45^{hi} macrophages whereas the EAE resistant p19KO mice had considerably fewer infiltrating T cells and macrophages. In addition, CD4⁻ CD11b⁺ CD45^{low} microglia numbers did not increase in p19KO mice after immunization. Moreover, CD4⁻ CD11b⁺CD45^{low} microglia numbers did not increase in p19KO mice after immunization and failed to up-regulate MHC-class II molecules. Thus, in the absence of IL-23, Th1 cells entered the CNS, but their presence did not lead to further recruitment of T cells, macrophages, or activation of resident microglia.

IX. IL-23 Directly Activates Macrophages to Product IL-1 beta and TNF.

IL-23 was tested for a direct effect on myeloid cells. IL-23 was administered, using IL-12 as a control cytokine, into the peritoneum of mice and analyzed peritoneal macrophage gene expression by quantitative real-time PCR (Table 6). Three hours after cytokine injection, IL-23 but not IL-12 treatment induced macrophages (F4/80⁺, CD11b⁺, CD11c³¹ B220⁻) to express IL-1beta and TNF mRNA. In contrast, both IL-12 and IL-23 increased the expression levels of CD40 and a range of other inflammatory molecules including matrix metalloproteases (MMP): MMP2, MMP7, MMP9, and inducible nitric oxide synthase (iNOS) (Table 6).

Methods relevant to the above work are as follows. C57BL/6 wild type mice were given no injection, 5 micrograms of IL-12, or 5 micrograms of IL-23 (i.p.). Three hours after the (i.p.) injection, F4/80⁺CD11b⁺CD11c⁻B220⁻ peritoneal macrophages were sorted and prepared for quantitative real time PCR analysis (Table 6). PCR analysis was used to measure expression of TNF, IL-1beta, and CD40 (Table 6).

The effect of anti-p40 antibody versus anti-IFNgamma antibody was studied. Mice were given no injection, injected with isotype antibody plus IL-23, or injected (i.p.) with a 10-fold molar excess of monoclonal antibodies against p40 (anti-p40 antibody) plus 5 micrograms of IL-23 (Table 7). Expression of TNF and IL-1beta were then assessed. Expression is relative to that of ubiquitin (1.0). The results demonstrate that anti-p40 antibody can block IL-23's stimulation of TNF and IL-1beta expression. Conversely, one-hour pre-treatment of mice with anti-IFNgamma antibody prior to IL-23 injection blocked elevated expression of CD40 but not of IL-1beta or TNF. IL-23 also induced expression of IL-1 beta and TNF but not CD40 in peritoneal macrophages in IFNgamma deficient mice (Table 7).

Mice were injected (i.p.) with 1 mg anti-IFNgamma antibody or isotype control monoclonal antibodies 1 hour before (i.p.) administration of 5 micrograms of IL-23. Three hours after IL-23 treatment, macrophages were isolated, sorted to purity, and prepared for PCR analysis of expression of TNF, IL-1beta, or CD40 (Table 8). IL-23 provoked increases of TNF and IL-1beta expression, where these increases were not dependent on the mouse's IFNgamma, that is, anti-IFNgamma antibody did not block the increase in IL-1 beta or TNF. In contrast, IL-23 provoked increases in CD40 expression, where this increase was shown to depend on the mouse's IFNgamma. Increase in other proinflammatory mediators, i.e., MMPs and iNOS, was IFNgamma dependent (Table 8).

TABLE 6
IL-23 versus IL-12, in stimulating expression of
TNF, IL-1beta, or CD40. Mice were treated, and expression
was assessed in isolated macrophages.

| | Expression by Taqman ® real time PCR. | | |
|---|---|---|---|
| | Ubiquitin = 1.0. | | |
| Treatment | TNF | IL-1beta | CD40 |
| naive | 100 | 400 | 700 |
| IL-12 | 250 | 550 | 3300 |
| IL-23 | 1600 | 4000 | 2000 |

TABLE 7
IL-23 versus [IL-23 plus anti-p40 antibody], in stimulating
expression of TNF or IL-1beta. Mice were treated, and expression
was assessed in isolated macrophages.

Expression by Taqman ® real time PCR.
Ubiquitin = 1.0.

| Treatment | TNF | IL-1beta |
|---|---|---|
| No injection | 300 | 300 |
| isotype + IL-23 | 1000 | 5750 |
| anti-p40 + IL-23 | 450 | 600 |

TABLE 8
IL-23, anti-IFN antibody, or [IL-23 plus anti-IFN antibody],
in stimulation of TNF, IL-1beta, or CD40 expression.
Mice were treated, and expression was assessed
in isolated macrophages.

| | Expression by Taqman ® real time PCR. | | |
|---|---|---|---|
| | Ubiquitin = 1.0. | | |
| Treatment | TNF | IL-1beta | CD40 |
| No injection | 275 | 1400 | 3750 |
| isotype mAb only | 250 | not determined | not determined |
| anti-IFNgamma only | 450 | not determined | not determined |
| isotype + IL-23 | 1450 | 9700 | 18500 |
| anti-IFNgamma + IL-23 | 2100 | 8800 | 4500 |

X. p19 Expression by Microglia and Macrophages in the CNS Increases with Onset of EAE.

Expression of IL-23, IL-12, and their receptors was measured in cells of the CNS, e.g., resident microglia and inflammatory macrophages. Microglia and inflammatory macrophages entering the CNS were sorted to purity and assessed for expression of the indicated subunits. Cells were isolated 2 days before clinical onset of EAE or 2 days after clinical onset when mice were severely affected (Table 9). Measurement of receptor subunit expression shows that microglia respond to IL-12, but not to IL-23, with EAE onset. The data also demonstrate that macrophages respond to both IL-23 and IL-12, with EAE onset. The post-onset increases in p19 expression by microglia and CNS macrophages, and the post-onset increases in IL-23R expression by CNS macrophages, demonstrate a role for IL-23 in mediating late-stage inflammation (Table 9).

TABLE 9
Expression of cytokine subunits and cytokine receptor
subunits in microglia and CNS macrophages, 2 days before
and 2 days after onset of EAE. Expression relative to
ubiquitin (1.0).

| Source of cells | | | | | | | |
|---|---|---|---|---|---|---|---|
| used for | | IL- | IL | IL- | | | |
| expression analysis. | | 23R | 12Rbeta1 | 12Rbeta2 | p19 | p40 | p35 |
| Microglia | before | <0.5 | 5 | 590 | 1.0 | 17 | 1.0 |
| | after | <0.5 | 550 | 360 | 7.0 | 38 | 1.2 |
| CNS | before | 1.0 | <5 | 280 | 1.3 | 24 | 1.0 |
| macro- | after | 20 | 320 | 490 | 9.0 | 17 | 19.0 |
| phages | | | | | | | |

XI. IL-17 Producing T Cell.

A new type of CD4⁺ T cell was identified: an IL-17-producing T cell. p19KO mice, or normal mice treated with an anti-p19 antibody, were found not to develop EAE. In normal, wild type mice primed with antigen, a population of antigen-specific CD4⁺ T cells which produced IL-17 were detected. These IL-17 producing cells were distinct from the IFNgamma producing CD4⁺ T cell population, i.e., TH1-type T cells. IL-17 producers were also detected in p35KO mice, that is, in mice lacking the p35 subunit of IL-12. However, IL-17 producing cells were absent in the p19KO mice suggesting that IL-23 is required for IL-17 production in vivo.

Methods for the preparation of different groups of mice, and the identification of strains of mice that generate or that cannot generate the unique IL-17 producing cells, were as follows. Wild type, IL-23p19KO (IL-23 deficient), IL-12p35KO (IL-12 deficient) and IL-12p40KO (both IL-12 and IL-23 deficient) mice on a C57BL/6 background were immunized (s.c.) with myelin oligodendrocyte glycoprotein (MOG) emulsified in complete freunds adjuvant, and with (i.v.) pertussis toxin. Draining lymph nodes (DLN) were removed at day 9 post-immunization, and mononuclear cells isolated, and stimulated for 3 hours with phorbol myristate acetate (PMA) (50 ng/ml), ionomycin (500 ng/ml) in the presence of Golgi-plug, then surface stained for CD4, permeabilized and intracellular stained for IFNgamma and IL-17. Plots were gated on alive CD4⁺ T cells.

In vitro studies using draining lymph node cells demonstrated that eliminating IL-23 inhibits or eliminates IL-17 producing cells, while adding IL-23 generates or stimulates IL-17 secretion, as determined by FACS analysis. The in vitro treatments with cytokine or antibodies were for 5 days. Draining lymph node (DLN) cells were isolated from antigen-primed normal wild type mice, and cultured in the presence of either rIL-12 or rIL-23. Analysis of the CD4⁺ T cells in the DLN cultures demonstrated that IL-12 promoted the development of IFNgamma producing cells, with loss of the IL-17 producing population. In contrast, IL-23 promoted the development of IL-17 producing cells, with loss of the IFNgamma producing population. Anti-p19 antibodies reduced IL-17 production but did not affect IFNgamma levels, whereas anti-p35 antibodies did not change IL-17 production. Taken together these results show that IL-23 selectively promotes the development of IL-17 producing CD4⁺ T cells.

In vitro-generated IL-17-producing cells were characterized in further detail, e.g., as to the ability of these cells to be converted to TH1-type cells. Using cultures previously treated with IL-23, to generate IL-17 producing cells, or cultures previously treated with IL-12, to generate TH1-type cells, each sample was split and further cultured with rIL-12 or rIL-23 to see whether these cytokines were able to regulate the generation of the IL-17 producing cells. Addition of rIL-23 to IFNgamma producing cells did not promote IL-17 production, and had no effect of the proportion of cells in that sample producing IFNgamma (in comparison to the IFNgamma+cells treated with rIL-12). However, addition of rIL-12 to IL-17 producing cells caused a dramatic reduction in IL-17 producing cells, and an increase in IFNgamma production (in comparison to the IL-17+cells treated with rIL-23). Overall these results suggest that the IL-12 down-regulates the IL-23-dependent production of IL-17, and may therefore play a protective role in late stage inflammation.

Methodologies used for the above study were as follows: Normal wild type SJL mice were immunized (s.c.) with proteolipid peptide (PLP) emulsified in complete Freund's adjuvant, and with (i.v.) pertussis toxin. Draining lymph nodes (DLN) were removed at day 9 post-immunization, and mononuclear cells isolated, and cultured in the presence of PLP (only for first 4 days) plus either rIL-12 or rIL-23 for 18 days (with additional IL-2 from day 7 onwards) to generate antigen-primed IFNgamma and IL-17 producing CD4⁺ T cells respectively. Each sample was split in two; half receiving rIL-12 and the other half receiving rIL-23, for a further 7 days of culture. Cells were stimulated for 3 hours with PMA/ionomycin in the presence of Golgi-plug, then surface stained for CD4, permeabilized, and intracellular stained for IFNgamma and IL-17. Plots were gated on alive CD4⁺ T cells.

IL-17-producing cells were further characterized by cell surface markers, e.g., CD45RB, and by cytokine secretion. Analysis of surface marker expression on the surface identified both the IL-17 producing cells and IFNgamma producing cells to express CD4⁺ CD62L^{lo} CD44^{hi}, indicative of an activated/effector memory T cells. However, the cells differed in their expression levels of CD45RB, which was much lower for the IL-17 producers than observed with the IFNgamma population. CD45RB is a marker commonly used to distinguish naïve and memory T cells, with CD4⁺ T cells observed to lose CD45RB expression as they progress from naïve to memory, suggesting that the IFNgamma producing CD4⁺ T cells are more naïve than their IL-17 producing counterparts (Annacker, et al. (2001) J. Immunol. 166:3008-3018).

Relevant methodologies were as follows: Normal wild type SJL mice were immunized (s.c.) with proteolipid peptide (PLP) emulsified in complete freunds adjuvant, and with (i.v.) pertussis toxin. Draining lymph nodes (DLN) were removed at day 9 post-immunization, and mononuclear cells isolated, and cultured in the presence of PLP (only for first 4 days) plus either rIL-12 or rIL-23 for 25 days (with additional IL-2 from day 7 onwards). Cells were stimulated for 3 hours with PMA/ionomycin in the presence of Golgi-plug, then surface stained for CD4 plus CD45RB, CD62L or CD44, permeabilized, and intracellular stained for either IFNgamma and IL-17. Plots were gated on alive CD4⁺ T cells.

IL-17-producing cells were characterized by expression of cytokines, cytokine receptor subunits (Table 10A), and other genes (Table 10B). Cells cultured with either IL-23 (IL-17 producers) or IL-12 (IFNgamma producers) were analyzed by Taqman® quantitative real-time PCR in comparison to naïve (not antigen-primed mice) and ex vivo (not cultured) DLN cells. As expected the cells driven with IL-23 had reduced IFNgamma mRNA message, elevated IL-17 and IL-75 mRNA expression. IL-75 is also known as IL-17F (see, e.g., Starnes, et al. (2001) J. Immunol. 167:4137-4140; Hurst, et al. (2002) J. Immunol. 169:443-453). In addition the IL-23 driven cells co-expressed IL-12Rbeta1 and IL-23R required for IL-23 receptor, but had low levels of IL-12Rbeta2 required for IL-12 signaling (Table 10A).

Methodologies relevant to the above study were as follows. Draining lymph nodes were harvested from naïve wild type SJL mice, and either immediately frozen in cell pellets (naive—PMA) or stimulated for 3 hours in the presence of PMA/ionomycin, then pelleted and frozen (naïve). For ex vivo and cultured cells, wild type SJL mice were immunized with PLP/CFA (s.c.) plus pertussis toxin (i.v.) Draining lymph nodes (DLN) were removed at day 9 post-immunization, and the mononuclear cells isolated. Cells were then either immediately stimulated for 3 hours PMA and ionomycin (ex vivo), or cultured in the presence of rIL-23 or rIL-12 for 11 days, prior to PMA/ionomycin stimulation and cell pelleting. RNA was extracted from the cell pellets, reverse transcribed into cDNA and used as a template for quantitative real-time PCR, with results normalized to the housekeeping gene, ubiquitin (Table 10A).

TABLE 10A
Gene expression of IL-17-producing cells versus
IFNgamma producing cells. Where indicated,
treatment with IL-23 or IL-12 was for 11 days and then
followed by treatment with PMA/ionomycin.

| | Taqman ® real time PCR analysis, relative to ubiquitin (1.0) | | | | | |
|---|---|---|---|---|---|---|
| | | | | IL- | | |
| Source of cell | IFNgamma | IL-17 | IL-75 | 12Rbeta1 | IL-12Rbeta2 | IL-23 |
| naïve (−PMA/iono.) | <2 | <2 | <2 | 10 | 100 | 4 |
| naïve (+PMA/iono.) | 1050 | 500 | 100 | 10 | 400 | 8 |
| ex vivo (+PMA/iono.) | 650 | 3000 | 1500 | 20 | 250 | 35 |
| +IL-23 (+PMA/iono.) | 750 | 25,000 | 7300 | 220 | 525 | 180 |
| +IL-12 (+PMA/iono.) | 2300 | 500 | 50 | 190 | 1800 | 20 |

TABLE 10B
Affymetrix Gene Chip ® analysis revealed genes that are
differentially increased with IL-23 treatment. The source of cells were
lymph node cells from PLP immunized mice, with ex vivo treatment
with IL-23 or IL-12. Data was analyzed by GeneSpring ® (Silicon
Genetics, Redwood City, CA). IL-75 expression was shown to be
specifically increased by IL-23, in a separate study not
involving the Gene Chip.

| | | Ratio of fold increase: |
|---|---|---|
| | | [IL-23-treated cells]/ |
| GenBank No. | Function | [IL-12 treated cells] |
| U18869 | Mitogen responsive P-protein | 49.4 |
| NM_011333; | Chemokine ligand 2 | 39.4 |
| M19681 | | |
| NM_010552 | IL-17 | 34.0 |
| NM_011281 | Transcription factor, RAR related | 30.4 |
| NM_007707 | Suppressor of cytokine signaling 3 | 22.9 |
| AA064471 | RIKEN cDNA 2310008N12 gene | 18.4 |
| NM_008362 | IL-1 receptor, type I | 17.5 |
| AI256158 | Glucosaminyltransferase I-branching enzyme | 16.1 |
| NM_008605 | Matrix metalloproteinase 12 | 15.0 |
| AW124225 | Expressed sequence AI848729 | 12.6 |
| NM_019471 | Matrix metalloproteinase 10 | 12.2 |
| AV296781 | EST. This EST contigs with MAX | 11.4 |
| | dimerization protein 1 Transcription factor. | |
| AA960140 | EST AA960140. This EST contigs with other | 11.2 |
| | probes annotated as carboxypeptidase E, | |
| | additionally with AK032306. | |
| AV223216 | Interleukin 1 receptor, type II | 11.4 |
| U55641 | Immunoglobin kappa chain V28 | 11.2 |
| AW120563 | MGC: 48196. IMAGE: 1514401. Contiged | 10.3 |
| | with XM_284368. | |
| AA003786 | AE binding protein 2 | 9.4 |
| NM_008250; | Transcription factor, H2.0-like homeo box | 8.2 |
| X58250 | | |
| AW047717 | Nedd4 WW binding protein 4 | 8.4 |
| AI849305 | IMAGE: 3590815 Contig to BC023404, | 7.2 |
| | AK078108, similar to protein tyrosine | |
| | phosphatase receptor type zeta, morphogenesis | |
| | of Purkinje cell dendrites in the developing | |
| | cerebellum | |
| NM_008535 | Transcription factor | 3.3 |
| NM_008543 | Transcription factor | 3.0 |
| NM_011345 | Adhesion molecule; ELAM-1 | 3.0 |
| NM_017373 | Transcription factor | 2.5 |
| NM_010751 | Transcription factor, MAX dimerization | 2.4 |
| | protein | |
| NM_012005 | Transcription factor | 2.4 |
| NM_013646 | Transcription factor | 2.1 |
| — | Interleukin-75 (IL-75) (see legend) | — |

Gene chip analysis demonstrated that the IL-17-producing cells expresses a number of genes not expressed by TH1-type T cells, where many of these genes are novel and previously uncharacterized. Cells were treated with IL-23, IL-12, or [IL-23 plus IL-12], followed by gene chip analysis to monitor mRNA expression. The results are described below and in Table 10B. In brief, gene chip analysis compares expressed genes from a sample, e.g., of cells or a tissue, with an array of pre-identified genes on a chip, where hybridization and specific binding of the expressed genes to the array on the chip enables identification of the expressed gene.

The present invention provides an IL-17 producing cell that expresses at least one gene, typically at least two genes, more typically at least three genes, most typically at least four genes, optimally at least five genes, more optimally at least six genes, and most optimally at least seven genes, ideally at least eight genes, more ideally at least nine genes, and most ideally at least ten genes, selected from IL-75 and Table 10B, normally by at least 2-fold greater with IL-23 treatment than with IL-12 treatment.

The present invention also provides an IL-17 producing cell that expresses at least one gene, typically at least two genes, more typically at least three genes, most typically at least four genes, optimally at least five genes, more optimally at least six genes, and most optimally at least seven genes, ideally at least eight genes, more ideally at least nine genes, and most ideally at least ten genes, selected from IL-75 and Table 10B, more normally by at least 4-fold, most normally by at least 8-fold, generally by at least 10-fold, more generally by at least 12-fold, preferably by at least 15-fold, more preferably by at least 20-fold, most preferably by at least 25-fold, optimally by at least 30-fold, more optimally by at least 35-fold, and most optimally by at least 40-fold, greater with IL-23 treatment than with IL-12 treatment.

Expression can be measured, e.g., by assessing levels of mRNA or of polypeptide. The invention provides an IL-17 producing cell, wherein the expression with stimulation with IL-23, as compared to stimulation with IL-12, is generally at least 2-fold, most generally at least 4-fold, most generally at least 10-fold, more typically at least 15-fold, most typically at least 20-fold, optimally at least 25-fold, more optimally at least 30-fold, most optimally at least 40-fold, and ideally by at least 80-fold. In comparisons of gene expression during stimulation with IL-23 or with IL-12, the source of cells can be, e.g., draining lymph node cells, PBMCs, or a substantially pure preparation of IL-17 producing CD4⁺ T cells.

The relevant methodology was as follows: Draining lymph nodes were harvested from wild type SJL mice immunized with PLP/CFA (s.c.) plus pertussis toxin i.v. Draining lymph nodes (DLN) were removed at day 9 post-immunization, and the mononuclear cells isolated. Cells were then either immediately stimulated for 3 hours PMA and ionomycin, ex vivo, or cultured in the presence of rIL-23 or rIL-12 for 11 days, prior to PMA/ionomycin stimulation and cell pelleting. RNA was extracted from the cell pellets, reverse transcribed into cDNA and used as a template for quantitative Affymetrix® gene chip analysis (Affymetrix, Santa Clara, Calif.).

With IL-23 treatment, 162 genes were upregulated by 5-fold or greater; with IL-12 treatment, 306 genes were upregulated by 5-fold or greater; while with with both [IL-23 and IL-12], 428 genes were upregulated by 5-fold or greater. Of the 306 genes specifically up-regulated in the IL-12 stimulated cells, nearly all were known genes with characterized functions, and were mainly anti-microbial/cytotoxic in their function, e.g., proteinases, granzymes, NK T cell genes, and genes with cytotoxic T cell functions and host-defense functions. Of the 162 genes specifically up-regulated in the IL-23 stimulated cells, IL-17 had relatively high gene expression, while other well expressed genes were identified as transcription factors and adhesion molecules. Taken together, these results demonstrate that DLN cells stimulated with IL-23 generates a novel population of cells, distinct from the IL-12 driven Th1 cell cultures, which display a divergent pattern of gene expression (Table 10B). The present invention provides methods for the inhibition of IL-17 producing cells, e.g., by an antibody to an adhesion protein or anti-sense DNA to a transcription factor specifically expressed by the IL-17 producing cell.

XII. Passive Transfer Technique with IL-17 Producing Cells Generates EAE.

The passive transfer technique was used to produce EAE in mice, where the results demonstrated a role of the IL-17 producing cells in the generation of autoimmune disorders, e.g., EAE or multiple sclerosis. Antigen-primed DLN cells were driven in vitro with either IL-23, to generate IL-17-producing cells, with or IL-12, to generate TH1-type cells, then passively transferred by injection (i.v.) into normal recipient wild type SJL mice.

The two groups of mice were examined to determine which cell population contributes to the development of EAE. Recipient mice injected with IL-17-producing cells developed EAE, with initial symptoms observed at days 7-8, an EAE score of 1.0 at day 10, a peak of disease at day 13 (EAE score of 1.9), with a decline found at day 15 (EAE score1.1). In contrast, mice injected with TH1-type cells, that is IL-12 driven IFNgamma producing CD4⁺ T cells, did not develop EAE, with no symptoms observed at any time-point (EAE score 0). These results indicate that the IL-17 producing CD4⁺ T cells are responsible for the development of EAE.

Relevant methodology was as follows: Normal recipient SJL mice were injected i.v. with either 2.5×10⁵ IFNgamma producing cells, or 1.5×10⁵ IL-17 producing cells. EAE score for each mouse was recorded daily, and averaged for each group. Mice that received IFNgamma producers did not develop any symptoms of EAE at any time-point during the study.

The severity of EAE disease relative to the number of cells injected into recipient mice was compared. Four different groups of mice were titrated with four different preparations of IL-17 producing cells. Each preparation of IL-17 producing cells contained a different ratio of [IL-17 producing cells]/[IFNgamma producing cells] (Table 11). 1.2×10⁶ CD4⁺ IL-17-producing T cells for passive transfer (based on calculation from intracellular cytokine staining results) gave severe EAE disease in the recipient mice. Reducing the number of IL-17 producing CD4⁺ T cells demonstrated reduced clinical scores in these recipient mice.

Therefore, these results show that the number of IL-17 producing CD4⁺ T cell passively transferred directly correlates with the severity of EAE disease displayed by the recipient SJL mice (Table 11).

The relevant methodology was as follows: Draining lymph node cells were isolated from wild type SJL mice at day 9 post-immunization with PLP/CFA+PTx. Cells were cultured for 10 days with either IL-23 or IL-12, and analyzed by intracellular cytokine staining to determine the proportion of cells producing IFNgamma, IL-17, and the overall cell number. Normal recipient SJL mice were then injected (i.v.) with the following numbers of these cultured IFNgamma producing cells, or IL-17 producing cells (Table 11).

TABLE 11
Identity of cell population used in passive transfer studies
from mouse groups 1-4.

| | | | | | Days of |
|---|---|---|---|---|---|
| | | IL-17 | IFNgamma | Peak EAE | peak |
| Mouse | Culture | producing | producing | disease | EAE |
| group | condition | cells | cells | score | score |
| 1 | IL-23 added | 12.0 × 10⁵ | 2.1 × 10⁵ | 4.9 | 9-10 |
| 2 | neutral | 3.0 × 10⁵ | 4.1 × 10⁵ | 4.3 | 11 |
| 3 | IL-23 added | 1.5 × 10⁵ | 4.6 × 10⁴ | 1.9 | 13 |
| 4 | IL-12 added | 0.2 × 10⁵ | 2.5 × 10⁵ | 0 | — |

Because of the finding that transfer of these IL-17 producing cells into normal recipient mice produced EAE, it was determined whether blocking the IL-17 produced by these cells with neutralizing antibodies against IL-17 could prevent the development of EAE in the recipient mice. Mice were administered either isotype control antibodies or a cocktail of two neutralizing anti-IL-17 antibodies, then the IL-17 producing cells were transferred into these mice (Table 12). Mice treated with isotype control plus IL-17⁺ cells showed a typical pattern on EAE progression, with initial clinical symptoms observed around day 7-8, and progression to peak of disease around days 12-13 days. Mice treated with anti-IL-17 mAbs prior to IL-17⁺ cell transfer showed a delay in EAE progression, with initial symptoms apparent at day 10, and peak of disease around day 15-16. These results suggest that the IL-17 produced by these cells does contribute to the development of EAE, however it is not the only cytokine/factor involved. Based on the Taqman® gene expression results shown earlier, it is possible that the high levels of TNF and IL-75 may also contribute to the development of EAE (Table 12).

The relevant methodology was as follows: Normal recipient SJL mice were injected (i.p.) with either isotype control mAb (25D2) at 100 micrograms/mouse, or anti-IL-17 mAbs (23E12 plus 1D10) at 50 micrograms each mAb/mouse. Mice were then injected (i.v.) with 5×10⁶ IL-17 producing cells. EAE score for each mouse was recorded daily, and averaged for each group (Table 12).

TABLE 12
Development of EAE by passive transfer, with treatment
with isotype control antibody (25D2) or
anti-IL-17 antibody (23E12 + 1D10).

| Antibody | EAE score on indicated day | | | | | | | | |
|---|---|---|---|---|---|---|---|---|---|
| treatment | Day4 | 5 | 6 | 7 | 8 | 9 | 10 | 11 | 12 |
| Isotype | 0 | ND | 0 | ND | 0.7 | 1.1 | 2.8 | 3.0 | 3.8 |
| control antibody | | | | | | | | | |
| Anti-IL-17 antibody | 0 | ND | 0 | ND | 0 | 0 | 0 | 0.9 | 2.1 |

XIII. IL-17 Producing Cells and Inflammatory Bowel Disorder (IBD).

IL-23 was found to be essential for chronic intestinal inflammation. The primary target of IL-23 was found to be a unique subset of tissue-homing memory T cells, identified as the “IL-17 producing cell.” Two strains of mice used as models of inflammatory bowel disorder (IBD) were studied, the IL-10KO mouse, which spontaneously develops a colitis that resembles Crohn's disease, and lymphocyte-deficient Rag KOmice, which develop colitis after reconstitution with CD4+T cells from IL-10KO mice. The intestinal disease that occurs in these models is initiated by excessive IFNgamma-producing cells, that is, by TH1-cells that are driven by IL-12. Thus, early treatment with anti-IFNgamma antibody or anti-p40 antibody (p40 is a subunit of IL-12) prevents the disease. Attempts to treat ongoing disease by treatment with anti-p40 antibody succeeded, but in contrast attempts to treat ongoing disease with anti-IFNgamma failed. Thus, any role of IL-12 (or its constituent p40 subunit) in producing IBD was independent from its ability to generate IFNgamma producing cells (see, e.g., Davidson, et al. (1998) J. Immunol. 161:3143-3149; Davidson, et al. (1996) J. Exp. Med. 184:241-251; Neurath, et al. (1995) J. Exp. Med. 182:1281-1290; Berg, et al. (1996) J. Clin. Invest. 98:1010-1012).

IL-23 plays a critical role in IBD. When IL-10KO mice were backcrossed with p19KO mice, the IL-10×p19 double KO mice, which lack p19 and lack the IL-23 heterodimer, were still disease free at 12 months of age (Table 13). Well before this time, the IL-10KO mice of the IBD disease model had developed colitis, i.e., by 3 months of age. At 12 months, half of the IL-10KO colony had died, and 100% of the survivors showed severe colitis, as determined by disease score, e.g., histological methods (Table 13). Photomicrographs of the descending colons from IL-10KO mice showed marked mucosal thickening and epithelial hyperplasia, where inflammation extended into the submucosa and tunica muscularis. IL-10×p 19 double KO mice did not show these pathological features.

TABLE 13
Inflammatory bowel disease score in IL-10KO mice,
p19KO mice, and IL-10 × p19KO mice.

| | Disease score | | |
|---|---|---|---|
| Strain of mouse | 3 months | 6 months | 12 months |
| IL-10 × p19KO mice | 1.6 | 1.0 | 1.7 |
| IL-10KO mice | 11.9 | 17.3 | 13.8 |
| p19KO mice | 0.8 | 0.8 | 0.6 |
| wild type | 0.4 | 0.4 | 0.3 |

The finding that IL-10×p19KO mice did not develop colitis raised the possibility that they are impaired in an ability to generate a pathologic TH1-type response. However, the studies of the present invention suggested otherwise. CD4⁺ T cells from IL-10KO and IL-10KO×p19KO mice secreted large amounts of IFNgamma (50-100 ng/10⁵ cells). For the IFNgamma secreting assays, sorted splenic CD4⁺CD45RB^{hi} naïve T cells isolated from the indicated mice were cultured on anti-CD3 antibody-coated plates in the presence of IL-12, for 4 days (Table 14). IL-12 was produced in similar amounts by LPS-stimulated macrophages from both strains of mice (2 to 6 ng/10⁶ cells; 72 h incubation), indicating that IL-10×p 19KO and IL-10KO mice were equally capable of generating IFNgamma producing TH1-type cells and that IL-23 is not required from TH1-type response or development (Table 14). Thus, it appeared that the absence of negative regulation by IL-10, with the IL-10 knockout, and the uncontrolled generation of TH I-type cells are merely predisposing factors, since mice that are also, that is, additionally, deficient in IL-23 were protected from colitis.

TABLE 14
Secretion of IFNgamma (ng/ml) and IL-12 (ng/ml).

| Strain of mouse | |
|---|---|
| | Secretion of IFNgamma from CD4⁺ T cells. |
| p19 × IL-10KO | 13.5 |
| IL-10KO | 2.9 |
| | Secretion of IL-12 from LPS-stimulated splenic |
| | macrophages. |
| p19 × IL-10KO | 2.8 |
| IL-10KO | 6.0 |

A T cell transfer model of colitis was studied to assess how IL-23 modulates IBD. RagKO mice were used as recipient mice for the passive transfer of T cells, because RagKO mice are devoid of T cells and B cells. RagKO mice develop colitis 10⁻¹² weeks after reconstitution with either naïve T cells (CD4⁺CD45RB^{high}) or with memory T cells (CD4+CD45RB^{low}) from diseased IL-10KO mice. However, recipient RagKO mice that were treated daily with IL-23 developed colitis after only 4 weeks (Table 15). The accelerated onset of colitis occurred regardless of whether IL-23 treated RagKO mice were reconstituted with naïve or memory T cells (Table 15). IL-23 treatment also led to spenomegaly and blood neutrophilia (4,800 cells/mm³ blood), while saline treated controls still had normal spleens and baseline neutrophil counts (1,500 cells/mm³ blood). In absence of reconstitution with T cells, continuous infusion of IL-23 did not result in colitis.

TABLE 15
Development of colitis with saline treatment or
IL-23 treatment, with reconstitution with naïve CD4⁺ T cells
or memory CD4⁺ T cells. After reconstitution, mice
received daily infusions with IL-23 or saline for 4 weeks, as
indicated. IL-23 treatment supports development of colitis at
an early time (accelerated onset), e.g., at t = 4 weeks
after cell reconstitution. Accelerated onset of colitis occurs
with reconstitution with either naïve T cells or
memory T cells taken from diseased IL-10KO mice.

| Treatment | | Disease score |
|---|---|---|
| No T cells | saline | 1.8 |
| transferred | IL-23 | 1.75 |
| Memory T cells | saline | 1.8 |
| used in | IL-23 | 7.7 |
| reconstitution | | |
| Naive T cells | saline | 1.9 |
| used in | IL-23 | 8.0 |
| reconstitution | | |

The mesenteric lymph nodes of IL-23 treated recipients had greatly expanded numbers of CD4⁺ T cells and CD11b⁻CD11c⁺F4/80⁻ dendritic cells. Saline treatment or IL-23 treatment was for four weeks (Table 16).

TABLE 16
Cells in mesenteric lymph nodes. IL-23 treatment of
RagKO mice increases CD4⁺ T cells and
CD11b⁻CD11c⁺ F4/80⁻ dendritic cells,
in mesenteric lymph nodes.

| | | CD11b⁻CD11c⁺ F4/80⁻ dendritic cells |
|---|---|---|
| | CD4⁺ T cells | (accessory cells) |
| Saline | 2.5 × 10⁴ cells | 4.0 × 10⁴ cells |
| treatment | | |
| IL-23 treatment | 24 × 10⁴ cells | 19 × 10⁴ cells |

The relative contributions of IL-23 and IL-12 to colitis was addressed (Table 17). RagKO mice are devoid of T cells and B cells. RagKO mice, reconstituted with naïve T cells (CD4⁺CD45RB^{high} T cells) develop low levels of colitis at t=4 weeks, and develop high level colitis at t=10⁻¹² weeks, after reconstitution with T cells (Table 17). With IL-23 infusion, the low level colitis found at 4 weeks is increased to a high level, and with IL-23 infusion, the high level colitis found at 12 weeks becomes still higher, demonstrating a role of IL-23 in colitis (Table 17).

Eliminating IL-12 by the p40KO technique completely prevents the low level signs found at 4 weeks, even where there is the additional infusion with IL-23, thus indicating that IL-12 is a component in colitis, especially in the early stages of colitis development (Table 17).

Eliminating IL-12 by the p40KO technique prevented the high level of colitis that had been expected at the 12 week time point, resulting in a low level colitis at t=12 weeks. Eliminating IL-12 by the p40KO method, but with an IL-23 infusion, resulted in a moderate level of colitis at t=12 weeks, again demonstrating that IL-12 is a component of colitis, but not a component that is absolutely necessary for the development of moderate colitis, under the conditions examined (Table 17).

Note that continuous infusion of IL-23 did not result in colitis in unreconstituted RagKO mice. In the reconstituted RagKO mice, IL-23 treatment resulted in splenomegaly and blood neutrophilia (4,800 cells/mm³ blood). The mesenteric lymph nodes (MLN) of IL-23-treated reconstituted RagKO mice contained increased numbers of CD4⁺ T cells and CD11b⁻CD11c⁺F4/80⁻ dendritic cells.

TABLE 17
Development of colitis in mice. All mice were RagKO mice.
Mice were either RagKO mice, or Rag × p40 double KO mice.
Reconstitution was with naïve CD4⁺ T cells.
Saline or IL-23 was administered as a daily infusion, for 4 or
12 weeks. Colitis could be induced even in the complete
absence of IL-12, i.e., in the RagKO × p40KO mice.

| Treatment | | Disease score at 4 weeks | Disease score at 12 weeks |
|---|---|---|---|
| RagKO | saline | 1.0 (low) | 7.8 (high) |
| | IL-23 | 6.0 (moderate to high) | 11.3 (very high) |
| RagKO × | saline | 0.2 (absent) | 1.0 (low) |
| p40KO | IL-23 | 0.2 (absent) | 4.2 (moderate) |

Real time PCR analysis of colon samples was performed to better define the actions of IL-23 (Table 18). PCR analysis was performed on samples from T cell transfer recipients treated with IL-23 (mice with colitis); saline treated controls (no colitis); and naïve controls (no cell transfer) (Table 18) The PCR results demonstrated an increase in expression of IL-17, as well as of other genes (see below). Note that IFNgamma can be made by accessory cells and T cells, but IL-17 is made only by T cells, e.g., by human and murine T cells with a memory/activated phenotype (Yao, et al. (1995) J. Immunol. 155:5483-5486; Shin, et al. (1999) Cytokine 11:257-266; Fossiez (1998) Int. Rev. Immunol. 16:541-551).

An influx of activated inflammatory macrophages occurred, as shown by increased expression of the relevant genes (Table 18). The genes indicating activated macrophages included IL-1beta, TNFalpha, nitric oxide synthase-2 (NOS2) (Table 18). An influx of granulocytes, as shown by the increase in relevant genes, e.g., myeloperoxidase and 12-lipoxygenase. Other increases were in genes controlling digestion of extracellular matrices and migration of cells to the mucosa, e.g., MCP-1, MIG, MMP-7, and MMP-12. Moreover, the PCR expression results also showed an increase in CD3 epsilon chain, indicating infiltration by donor CD4⁺ T cells (Table 18).

IL-23 added to memory T cells provoked an increase in expression of IL-17 and IL-6, relative to responses to added IL-2 and IL-12, but IL-23 did not provoke increases in TNF or in IFNgamma (Table 19). Naïve T cells do not express IL-23R, and separate experiments showed that IL-23 had no effect on gene expression by naïve T cells (data not shown). The greater tendency of IL-23 to stimulate IL-17 and IL-6 production, as compared to the relatively low stimulatory effects of IL 2 and IL-12, selectively implicates IL-23 in disorders dependent on IL-17 and IL-6, e.g., inflammatory bowel disorders (see, e.g., Yamamoto, et al. (2000) J. Immunol. 164:4878-4882; Atreya, et al. (2000) Nature Med. 6:583-588; Ito, et al. (2002) J. Gastroenterol. 37 Suppl. 14:56-61., Gross, et al. (1992) Gastroenterol. 102:514-519; Fujino (2003) Gut 52:65-70; Hata, et al. (2002) Am. J. Physiol. Gastrointest. Liver Physiol. 282:G1035-G1044).

TABLE 18
Taqman ® real time PCR analysis of gene expression
in colon, relative to ubiquitin (1.0). Tissue samples were from
RagKO mice reconstituted with or without memory CD4⁺
T cells from IL-10KO mice, where the mice were treated as
indicated (4 week treatment). NA means data not available.
Treatment of mouse before tissue analysis

| | naïve (no cell | 4 weeks saline | 4 weeks IL-23 |
|---|---|---|---|
| Expression of: | transfer) | treatment | treatment |
| IL-1beta | 5 | 4 | 25 |
| TNFalpha | 7.5 | 7.5 | 55 |
| IFNgamma | 0.1 | 0.3 | 3.8 |
| IL-17 | <0.05 | 0.12 | 0.80 |
| MCP-1 | 5 | 2.5 | 38 |
| MIG | 50 | 75 | 900 |
| CD3 | 1 | 4 | 21 |
| NOS-2 | 15 | 20 | 550 |
| MMP-7 | NA | NA | increased |
| MMP-12 | NA | NA | increased |

TABLE 19
Taqman ® real time PCR analysis of expression of
IL-6, 1L-17, TNF, or IFNgamma in CD4⁺ memory
activated T cells from IL-10KO mice. All T cells were exposed
to anti-CD3 antibody.

| | Cytokine added to CD4⁺CD45RB^{low} memory T cells | | |
|---|---|---|---|
| | from IL 10KO mice. | | |
| | IL-2 | IL-2 | IL-23 |
| | Expression of IL-6, IL-17, TNF, or | | |
| Expression of: | IFNgamma by Taqman ® analysis | | |
| 1L-6 | 10 | 8 | 125 |
| 1L-17 | 120 | 180 | 510 |
| TNF | 1550 | 1000 | 1250 |
| IFNgamma | 1550 | 2400 | 1600 |

IL-23 stimulatory activity contrasted with IL-12 activity. IL-23-treatment stimulates IL-17 production to a much greater extant than IL-12-treatment. IL-12 stimulates IFNgamma production and IL-23 inhibits IFNgamma production, in studies of memory T cells from IL-10×p19 double KO mice, or memory T cells from IL-10KO mice (Table 20). Thus, IL-23 tended to be correlated with increased IL-17 production, while IL-12 tended to be correlated with increased IFNgamma production (Table 20).

The highest levels of IL-17 were induced by IL-23 in cells from IL-10KO mice, with moderate amounts of IL-17 from IL-10×p19 double KO mice, and no detectable IL-17 from cells from wild type mice and from p19KO mice (Table 20).

TABLE 20
Expression of IL-17, IFNgamma, and IL-4 by
memory CD4⁺ T cells isolated
from four strains of mice. Cells were exposed to
media only, or stimulated with IL-12, or IL-23.
Cell incubations were in the presence of plate-bound
anti-CD3 antibody. Expression was determined by ELISAs.

| | Additive to cell incubation mixtures | | | | | | | | |
|---|---|---|---|---|---|---|---|---|---|
| | none | IL-12 | IL-23 | none | IL-12 | IL-23 | none | IL-12 | IL-23 |
| | IL-17 expression | | | IFNgamma | | | IL-4 expression | | |
| | (ng/ml) | | | expression (ng/ml) | | | (ng/ml) | | |
| IL-10 × | 0.4 | 0.2 | 2.0 | 70 | 120 | 35 | 3.8 | 2.75 | 2.8 |
| p19KO | | | | | | | | | |
| IL- | 0.9 | 0.8 | 6.7 | 90 | 135 | 80 | 2.4 | 1.15 | 2.1 |
| 10KO | | | | | | | | | |
| p19KO | <0.1 | <0.1 | <0.1 | <0.1 | 10 | 5 | 1.1 | 0.75 | 0.8 |
| wild | <0.1 | <0.1 | <0.1 | <0.1 | 12 | 5 | 2.0 | 1.75 | 1.4 |
| type | | | | | | | | | |

Cell proliferation is another measure of inflammatory disorders, in addition to disease score (see, e.g., Tables 15 & 17), T cell number within lymph nodes (see, e.g., Table 16), and on gene expression (see, e.g., Tables 18-20). IL-23 was tested for its effect on cell proliferation. IL-23 dependent cell proliferation in the presence of anti-IL-2 antibody was tested on memory T cells from: (1) IL-10×p19KO mice; (2) IL-10KO mice; (3) p19KO mice; and (4) wild type mice. Memory cells from IL-10KO mice were the best responders (22,000 cpm tritium), while cells from IL-10×p19KO mice responded next best (8,000 cpm tritium). Little proliferation was shown by cells from p19KO mice or wild type mice. All proliferation assays were in the presence of anti IL 2 antibody.

A novel type of IL-17 producing T cell was found to have the following two properties: (1) Only diseased IL-10KO mice had large numbers of the IL-17 producing T cells; and (2) The IL-17 producing T cells were negative for IFNgamma, and thus were not classical TH1-type T cells, and were negative for IL-4, and thus were not classical TH2-type T cells. Here, expression of IFNgamma and IL-4 was determined by intracellular cytokine staining of memory T cells. In short, staining showed that the cells a subset distinct from classical TH1 and TH2-type memory cells. This particular subset can occur in the absence of endogenous IL-23 production, as indicated by the small, yet existent, amount of cells in the IL-10×p19KO mice. However, FACS analysis demonstrated that IL-23 is required for optimal expansion and IL-17 production (data not shown).

The present study examined the effect of treating mice exposed to conditions that induce colitis, with anti-IL-6 antibodies, anti-IL-17 antibodies, or both antibodies. IL-6 has been associated with bowel inflammation, while the role of IL-17 has been unclear (see, e.g., Yamamoto, et al., supra; Atreya, et al., supra; Ito, et al., supra, Gross, et al., supra; Fujino, supra; Hata, et al., supra). T cell reconstituted recipient mice were treated with IL-23 in order to induce colitis. During treatment to induce colitis, the indicated antibody or antibodies was also administered. Isotype control antibody, anti-IL-6 antibody, anti-IL-17 antibody, or both anti-IL-6 and anti-IL-17, were administered. Anti-IL-17 antibody alone, as well as the combination of anti-IL-17 antibody and anti-IL-6 antibody, improved the disease score (Table 21).

The methodology was as follows: Recipient mice were dosed (i.p.) with the indicated antibody or antibodies (2 mg/mouse) one day prior to T cell reconstitution (Table 21). RagKO mice were reconstituted with sorted splenic CD4⁺CD45RB^{hi} (naïve) T cells (5×10⁵ cells/mouse) from diseased IL-10KO mice, and treated daily with 1 microgram of IL-23 per mouse. Subsequent doses of the indicated antibody or antibodies were administered weekly for six weeks (Table 21).

TABLE 21
Improvement of IBD disease score with antibody treatment.
RagKO mice were reconstituted with T cells and treated with
IL-23 (conditions that provoke colitis), but also treated with the
indicated antibody or antibodies.

| Treatment | IBD disease score |
|---|---|
| Isotype antibody | 10.0 |
| Anti-IL-6 antibody + anti-IL-17 antibody | 5.2 |
| Anti-IL-6 andibody | 7.5 |
| Anti-IL-17 antibody | 6.8 |

What is claimed is:

1. A method of treating an IL-23 mediated disorder comprising administering an effective amount of an:
a) agonist of IL-23; or
b) antagonist of IL-23.

2. The method of claim 1, wherein the disorder is a:
a) gastrointestinal disorder; or
b) nervous system disorder.

3. The method of claim 1, wherein the agonist or antagonist specifically binds to a polypeptide or nucleic acid of:
a) p19; or
b) IL-23R.

4. The method of claim 1, wherein the agonist or antagonist comprises a:
a) nucleic acid; or
b) small molecule.

5. The method of claim 4, wherein the nucleic acid comprises:
a) anti-sense nucleic acid; or
b) small interfering RNA (siRNA).

6. The method of claim 1, wherein the agonist or antagonist comprises:
a) an antigen binding fragment of an antibody; or
b) a soluble receptor derived from IL-23R.

7. The method of claim 6, wherein the agonist or antagonist is:
a) a polyclonal antibody;
b) a monoclonal antibody;
c) a humanized antibody or binding fragment thereof;
d) an Fab, Fv, or F(ab′)₂ fragment;
e) a peptide mimetic of an antibody;
f) detectably labeled.

8. The method of claim 2, wherein treatment is with an antagonist of IL-23 and the nervous system disorder comprises a:
a) central nervous system (CNS) disorder; or
b) peripheral nervous system (PNS) disorder.

9. The method of claim 1, wherein treatment is with an antagonist of IL-23 and the condition or disorder comprises:
a) multiple sclerosis;
b) neuropathic pain;
c) amyotrophic lateral sclerosis (ALS);
d) ischemic brain injury; or
e) inflammatory bowel disorder.

10. The method of claim 9, wherein the inflammatory bowel disorder comprises:
a) Crohn's disease;
b) ulcerative colitis;
c) celiac disease;
d) mucosal thickening;
e) epithelial hyperplasia;
f) inflammation of the submucosa or tunica muscularis; or
g) infiltration by granulocytes or macrophages.

11. The method of claim 1, wherein the agonist or antagonist if IL-23 is co-administered with an agonist or antagonist of:
a) IL-12;
b) interferon-gamma (IFNgamma);
c) IL-6;
d) IL-17; or
e) IL-10.

12. The method of claim 2, wherein the nervous system disorder is exacerbated by an antagonist of:
a) IL-12; or
b) IFNgamma.

13. The method of claim 2, wherein the nervous system disorder:
a) comprises an increase in microglial expression of p19;
b) comprises an increase of CNS macrophage expression of IL-23R or p19; or
c) can be generated in human or animal subject by administration of exogenous IL-17 producing cells to the subject.

14. The method of claim 1, wherein treatment with the antagonist of IL-23 inhibits activation of a resident microglial cell.

15. The method of claim 14, wherein the:
a) microglial cell is CD11b⁺CD45^{low}; or
b) activation comprises up-regulation of MHC-Class II.

16. The method of claim 1, wherein the antagonist of IL-23 inhibits:
a) expression of IL-1beta by a macrophage;
b) expression of tumor necrosis factor (TNF) by a macrophage; or
c) infiltration of a macrophage into the central nervous system (CNS).

17. The method of claim 16, wherein the macrophage is:
a) F4/80⁺;
b) CD11b⁺;
c) CD11c⁻; or
d) B220⁻.

18. A purified or isolated IL-17 producing CD4⁺ T cell that upon treatment with IL-23 has a 10-fold higher expression of at least one gene of Table 10B when compared to treatment with IL-12.

19. The IL-17 producing T cell of claim 18 that is:
a) CD62L^{lo}CD44^{hi}; or
b) CD45RB^{lo}.

20. A method of generating the IL-17 producing CD4⁺ T cell of claim 18, comprising contacting a T cell with a substantially pure preparation of IL-23 or an agonist thereof.
